(19) United States
(12) Patent Application Publication
(10) Pub. No.: US 2025/0170093 A1
(43) Pub. Date: May 29, 2025

(54) ENDOPLASMIC RETICULUM PROTEIN HYOU1-BINDING COMPOUND FOR INHIBITING ENDOPLASMIC RETICULUM-MITOCHONDRIAL INTERACTION AND USES THEREOF

(71) Applicant: UIF (University Industry Foundation), Yonsei Univeristy, Seoul (KR)

(72) Inventors: Ho Jeong KWON, Seoul (KR); Min Jeong KO, Seoul (KR)

(73) Assignee: UIF (University Industry Foundation), Yonsei University, Seoul (KR)

(21) Appl. No.: 18/958,873

(22) Filed: Nov. 25, 2024

(30) Foreign Application Priority Data
Nov. 28, 2023 (KR) 10-2023-0167827
Nov. 13, 2024 (KR) 10-2024-0161191

(51) Int. Cl. A61K 31/343; A61P 9/10; A61P 9/12

(52) U.S. Cl. CPC A61K 31/343; A61P 9/10; A61P 9/12

(57) ABSTRACT

The present disclosure relates to endoplasmic reticulum protein HYOU1-binding compounds for inhibiting endoplasmic reticulum-mitochondrial interactions and uses thereof.

The features and advantages of the present disclosure may be summarized as follows:
(a) The present disclosure provides a compound that can bind to the endoplasmic reticulum protein HYOU1 and inhibit endoplasmic reticulum-mitochondrial interactions increased by oxLDL (oxidized low-density lipoprotein).
(b) The present disclosure provides a composition capable of preventing, ameliorating or treating autophagy disorder-related diseases, particularly arteriosclerosis or pulmonary hypertension, which contains the above-described compound as an active ingredient.
(c) Since the composition of the present disclosure reduces intracellular lipids by inducing autophagy in atherosclerosis-induced tissues, it can be useful used in the amelioration or treatment of atherosclerosis.
(d) Since the composition of the present disclosure reduces smooth muscle cell proliferation by inducing autophagy in smooth muscle cells in which contraction is induced by TGF-β, it can be usefully used in the amelioration or treatment of pulmonary arterial hypertension.

### BACKGROUND

### 1. Field

The present disclosure relates to endoplasmic reticulum protein HYOU1-binding compounds for inhibiting endoplasmic reticulum-mitochondrial interactions and uses thereof.

### 2. Description of the Related Art

Cell organelles are dynamic, membrane-enclosed systems that communicate with each other to maintain cellular homeostasis and perform their own functions. The endoplasmic reticulum (ER) is the largest organelle in the cell and is involved in protein synthesis, transport and folding, and lipid synthesis. In addition, the ER, which stores calcium in cells, interacts with various organelles, including mitochondria, Golgi apparatuses, lysosomes, peroxisomes, etc. The ER is also involved in Ca²⁺ and lipid transport, mitochondrial function, autophagy, etc. IP₃R-Grp75-VDAC1 is one of the key complexes of ER-mitochondrial contacts and calcium transport. Recent studies have shown that mitochondria-associated membranes (MAMs), where mitochondria are in contact with the endoplasmic reticulum, are also implicated in autophagy.

On the other hand, the increase in oxidized low-density lipoprotein (oxLDL) is a major contributor to atherosclerotic pathology in patients with diabetes and hypercholesterolemia. OxLDL causes dysfunction of endothelial cells (ECs) and disrupts vascular homeostasis. Sanjiu Yu et al. reported that, in atherosclerosis, oxLDL delivers excess calcium to mitochondria via an increase in MAMs, resulting in oxLDL-induced vascular endothelial cell death.

In addition, a recent study reported that autophagy is induced by reduced endoplasmic reticulum-mitochondrial interaction. Autophagy is the process of breaking down dysfunctional organelles or proteins for use as an energy source for a cell. It is activated in situations of cellular stress. Therefore, abnormalities in autophagic activity can lead to metabolic diseases, immune diseases, vascular diseases, etc.

Therefore, the development of compounds that exhibit anti-atherosclerotic and/or antihypertensive activity by modulating the MAMs, more specifically, binding to the endoplasmic reticulum protein HYOU1, thereby inhibiting the contact and interaction of the endoplasmic reticulum with mitochondria, inducing autophagy, is drawing attentions.

A number of literatures and patent documents are referred to and cited throughout this specification. The disclosures of the cited literatures and patent documents are incorporated herein by reference in their entirety for a more complete description of the present disclosure and the technical field to which it belongs.

### REFERENCES OF RELATED ART

### Non-Patent Documents

(Non-patent document 1) Phillips, M. J. & Voeltz, G. K. Structure and function of ER membrane contact sites with other organelles. Nature Reviews Molecular Cell Biology 17, 69-82 (2016).
(Non-patent document 2) Bravo-Sagua, R. et al. Organelle communication: signaling crossroads between homeostasis and disease. The International Journal of Biochemistry & Cell Biology 50, 55-59 (2014).
(Non-patent document 3) Jing, J., Liu, G., Huang, Y. & Zhou, Y. A molecular toolbox for interrogation of membrane contact sites. The Journal of Physiology 598, 1725-1739 (2020).
(Non-patent document 4) Ni, L. & Yuan, C. The mitochondrial-associated endoplasmic reticulum membrane and its role in diabetic nephropathy. Oxidative Medicine and Cellular Longevity 2021 (2021).
(Non-patent document 5) Gordaliza-Alaguero, I., Canto, C. & Zorzano, A. Metabolic implications of organelle-mitochondria communication. EMBO Reports 20, e47928 (2019).
(Non-patent document 6) Basso, V., Marchesan, E. & Ziviani, E. A trio has turned into a quartet: DJ-1 interacts with the IP₃R-Grp75-VDAC complex to control ER-mitochondria interaction. Cell Calcium 87, 102186 (2020).
(Non-patent document 7) Xu, H. et al. IP₃R-Grp75-VDAC1-MCU calcium regulation axis antagonists protect podocytes from apoptosis and decrease proteinuria in an adriamycin nephropathy rat model. BMC Nephrology 19, 1-11 (2018).
(Non-patent document 8) Gomez-Suaga, P. et al. The ER-mitochondria tethering complex VAPB-PTPIP51 regulates autophagy. Current Biology 27, 371-385 (2017).
(Non-patent document 9) Thoudam, T. et al. PDK4 augments ER-mitochondria contact to dampen skeletal muscle insulin signaling during obesity. Diabetes 68, 571-586 (2019).
(Non-patent document 10) Lee, H. J. et al. Urolithin A suppresses high glucose-induced neuronal amyloidogenesis by modulating TGM2-dependent ER-mitochondria contacts and calcium homeostasis. Cell Death & Differentiation 28, 184-202 (2021).
(Non-patent document 11) Wang, C. et al. FUNDC1-dependent mitochondria-associated endoplasmic reticulum membranes are involved in angiogenesis and neoangiogenesis. Nature Communications 12, 2616 (2021).
(Non-patent document 12) Angebault, C. et al. ER-mitochondria cross-talk is regulated by the Ca²⁺ sensor NCS1 and is impaired in Wolfram syndrome. Science Signaling 11, eaaq1380 (2018).
(Non-patent document 13) Filipe, A. et al. Defective endoplasmic reticulum-mitochondria contacts and bioenergetics in SEPN1-related myopathy. Cell Death & Differentiation 28, 123-138 (2021).
(Non-patent document 14) Poznyak, A. V. et al. Overview of oxLDL and its impact on cardiovascular health: focus on atherosclerosis. Frontiers in Pharmacology, 2248 (2021).
(Non-patent document 15) Yu, S. et al. PACS2 is required for oxLDL-induced endothelial cell apoptosis by regulating mitochondria-associated ER membrane formation and mitochondrial Ca²⁺ elevation. Experimental Cell Research 379, 191-202 (2019).
(Non-patent document 16) Ko, M., Oh, G. T., Park, J. & Kwon, H. J. Extract of high hydrostatic pressure-treated danshen (Salvia miltiorrhiza) ameliorates atherosclerosis via autophagy induction. BMB Reports 53, 652 (2020).
(Non-patent document 17) Lomenick, B. et al. Target identification using drug affinity responsive target stability (DARTS). Proceedings of the National Academy of Sciences 106, 21984-21989 (2009).
(Non-patent document 18) Chang, J., Kim, Y. & Kwon, H. Advances in identification and validation of protein targets of natural products without chemical modification. Natural Product Reports 33, 719-730 (2016).
(Non-patent document 19) Rao, S. et al. Biological function of HYOU1 in tumors and other diseases. OncoTargets and Therapy, 1727-1735 (2021).
(Non-patent document 20) Lee, A. S. Glucose-regulated proteins in cancer: molecular mechanisms and therapeutic potential. Nature Reviews Cancer 14, 263-276 (2014).
(Non-patent document 21) De Smet, P. et al. Xestospongin C is an equally potent inhibitor of the inositol 1,4,5-trisphosphate receptor and the endoplasmic-reticulum Ca²⁺ pumps. Cell Calcium 26, 9-13 (1999).
(Non-patent document 22) Cieri, D. et al. SPLICS: a split green fluorescent protein-based contact site sensor for narrow and wide heterotypic organelle juxtaposition. Cell Death & Differentiation 25, 1131-1145 (2018).
(Non-patent document 23) Vallese, F. et al. An expanded palette of improved SPLICS reporters detects multiple organelle contacts in vitro and in vivo. Nature Communications 11, 6069 (2020).
(Non-patent document 24) Sergin, I. et al. Exploiting macrophage autophagy-lysosomal biogenesis as a therapy for atherosclerosis. Nature Communications 8, 15750 (2017).
(Non-patent document 25) Wang, L. et al. Pdcd4 deficiency enhances macrophage lipoautophagy and attenuates foam cell formation and atherosclerosis in mice. Cell Death & Disease 7, e2055-e2055 (2016).
(Non-patent document 26) Grootaert, M. O. et al. Defective autophagy in vascular smooth muscle cells accelerates senescence and promotes neointima formation and atherogenesis. Autophagy 11, 2014-2032 (2015).
(Non-patent document 27) Vion, A.-C. et al. Autophagy is required for endothelial cell alignment and atheroprotection under physiological blood flow. Proceedings of the National Academy of Sciences 114, E8675-E8684 (2017).
(Non-patent document 28) Kim, D. et al. Activation of mitochondrial TUFM ameliorates metabolic dysregulation through coordinating autophagy induction. Commun Biol 4, 1, doi: 10.1038/s42003-020-01566-0 (2021).
(Non-patent document 29) Hwang, H.-Y., Shim, J. S., Kim, D. & Kwon, H. J. Antidepressant drug sertraline modulates AMPK-MTOR signaling-mediated autophagy via targeting mitochondrial VDAC1 protein. Autophagy 17, 2783-2799 (2021).
(Non-patent document 30) Kim, D. et al. FK506, an immunosuppressive drug, induces autophagy by binding to the V-ATPase catalytic subunit A in neuronal cells. Journal of Proteome Research 16, 55-64 (2017).
(Non-patent document 31) Hwang, H.-Y. et al. Autophagic inhibition via lysosomal integrity dysfunction leads to antitumor activity in glioma treatment. Cancers 12, 543 (2020).
(Non-patent document 32) Lorentzen, L. G., Hansen, G. M., Iversen, K. K., Bundgaard, H. & Davies, M. J. Proteomic Characterization of Atherosclerotic Lesions In Situ Using Percutaneous Coronary Intervention Angioplasty Balloons-Brief Report. Arteriosclerosis, Thrombosis, and Vascular Biology 42, 857-864 (2022).
(Non-patent document 33) Liu, Z. et al. Cryptotanshinone, an orally bioactive herbal compound from danshen, attenuates atherosclerosis in apolipoprotein E-deficient mice: Role of lectin-like oxidized LDL receptor-1 (LOX-1). British Journal of Pharmacology 172, 5661-5675 (2015).

### SUMMARY

HYOU1 (hypoxia-upregulated protein 1), also known as GRP170, is an ER-resident chaperone that functions in protein quality control within the ER. HYOU1 is also involved in the regulation of the unfolded protein response (UPR) signaling pathway, which is activated in response to ER stress. HYOU1 is known to be upregulated in hypoxic conditions induced by cancer and other pathological conditions, helping cells adapt to stress.

The inventors have made consistent efforts to identify novel compounds that modulate MAMs to interfere with the interaction of the endoplasmic reticulum, which is an intracellular organelle, with mitochondria. As a result, they have found that HYOU1 is a target that can efficiently inhibit the interaction of cell organelles in atherosclerosis and hypertension, and have completed the present disclosure by confirming that cryptotanshinone (CTS) binds to the HYOU1, inhibits ER-mitochondrial interaction, induces autophagy, and exhibits anti-atherosclerotic and/or antihypertensive activity.

Accordingly, the present disclosure is directed to providing a pharmaceutical composition for preventing or treating autophagy disorder-related diseases, which contains cryptotanshinone (CTS) or a pharmaceutically acceptable salt thereof as an active ingredient.

The present disclosure is also directed to providing a food composition for preventing or ameliorating autophagy disorder-related diseases, which contains cryptotanshinone (CTS) or a sitologically acceptable salt thereof as an active ingredient.

The present disclosure is also directed to providing a composition for activating autophagy in vitro, which contains cryptotanshinone (CTS) as an active ingredient.

The present disclosure is also directed to providing a composition for inhibiting endoplasmic reticulum-mitochondrial interactions in vitro, which contains cryptotanshinone (CTS) as an active ingredient.

The present disclosure provides a pharmaceutical composition for preventing or treating autophagy disorder-related diseases, which contains cryptotanshinone (CTS) or a pharmaceutically acceptable salt thereof as an active ingredient.

In an exemplary embodiment of the present disclosure, the CTS may have one or more characteristics selected from 1) to 4):
1) inhibition of calcium transfer from the endoplasmic reticulum to mitochondria;
2) increase of lysosomal activity;
3) increase of autophagic activity; and
4) increase of distance between the endoplasmic reticulum and mitochondria.

In an exemplary embodiment of the present disclosure, the CTS may bind to HYOU1 (hypoxia-upregulated protein 1) to induce autophagy.

In an exemplary embodiment of the present disclosure, the autophagy disorder-related disease may be a disease caused by accumulation of abnormal proteins induced by reduced intracellular autophagy or a degenerative disease.

In an exemplary embodiment of the present disclosure, the disease caused by the accumulation of abnormal proteins or degenerative disease may be any one selected from arteriosclerosis, pulmonary hypertension, Alzheimer's disease, Parkinson's disease, type 2 diabetes, amyotrophic lateral sclerosis, dialysis-related amyloidosis, cystic fibrosis, sickle cell anemia, Huntington's disease, Creutzfeldt-Jakob disease, Lewy body dementia, inclusion body myositis, cerebral amyloid angiopathy, traumatic brain injury, frontotemporal dementia, progressive supranuclear palsy, corticobasal degeneration, Pick's disease, and argyrophilic grain disease.

In an exemplary embodiment of the present disclosure, the arteriosclerosis may be atherosclerosis.

In an exemplary embodiment of the present disclosure, the composition may reduce intracellular lipids by inducing autophagy.

In an exemplary embodiment of the present disclosure, the pulmonary hypertension may be pulmonary arterial hypertension.

The present disclosure also provides a food composition for preventing or ameliorating of autophagy disorder-related diseases, which contains cryptotanshinone (CTS) or a sitologically acceptable salt thereof as an active ingredient.

The present disclosure also provides a composition for activating autophagy in vitro, which contains cryptotanshinone (CTS) as an active ingredient.

The present disclosure also provides a composition for inhibiting endoplasmic reticulum-mitochondrial interactions in vitro, which contains cryptotanshinone (CTS) as an active ingredient.

The features and advantages of the present disclosure may be summarized as follows:
(a) The present disclosure provides a compound that can bind to the endoplasmic reticulum protein HYOU1 and inhibit endoplasmic reticulum-mitochondrial interactions increased by oxLDL (oxidized low-density lipoprotein).
(b) The present disclosure provides a composition capable of preventing, ameliorating or treating autophagy disorder-related diseases, particularly arteriosclerosis or pulmonary hypertension, which contains the above-described compound as an active ingredient.
(c) Since the composition of the present disclosure reduces intracellular lipids by inducing autophagy in atherosclerosis-induced tissues, it can be useful used in the amelioration or treatment of atherosclerosis.
(d) Since the composition of the present disclosure reduces smooth muscle cell proliferation by inducing autophagy in smooth muscle cells in which contraction is induced by TGF-β, it can be usefully used in the amelioration or treatment of pulmonary arterial hypertension.

### BRIEF DESCRIPTION OF THE DRAWINGS

FIGS. 1A-1H show the effect of oxLDL on mitochondrial homeostasis and autophagy in HUVECs. FIG. 1A shows that cell proliferation is inhibited when HUVECs are treated with oxLDL. FIG. 1B shows the morphology of mitochondria in HUVECs treated with oxLDL. FIG. 1C shows the level of MitoROS in HUVECs treated with oxLDL (scale bar=20 μm). FIG. 1D shows a result of measuring mitochondrial calcium content in HUVECs treated or untreated with Xesto C (xestospongin C), which is an IP3R antagonist, after treating with oxLDL. FIG. 1E shows SPLICS (ER-mitochondria split-GFP) assay images indicating the interaction between the endoplasmic reticulum and mitochondria in oxLDL-treated HUVECs. HUVECs were transfected with a SPLICS plasmid and, after treating with oxLDL, the area (%) of GFP fluorescence relative to the total area per cell was measured. FIG. 1F shows PLA assay images indicating the interaction between cell organelles in oxLDL-treated HUVECs (scale bar=20 μm). FIG. 1G shows western blot assay images indicating SQSTM1 and LC3 protein expression levels in oxLDL-treated HUVECs. FIG. 1H shows confocal fluorescence microscopic images of HUVECs treated with oxLDL after transfection with the mRFP-GFP-LC3 vector. The yellow dots indicate autophagosomes and the red dots indicate autolysosomes.

FIGS. 2A-2G show that CTS attenuates atherosclerosis by inducing autophagy in ApoE knockout mice. FIG. 2A shows the images of RAW264.7 macrophages treated with HDE (danshen extract), Tan II-A, DHT or CTS, respectively, along with oxLDL and then stained with Oil Red O (scale bar=20 μm). FIG. 2B shows the showing images of plaques in the aorta harvested from an atherosclerosis-induced animal model to which CTS was administered, observed after staining with Oil Red O, and a result of quantifying the ratio of lesion area (Oil Red O-stained area) relative to the total aortic area. FIG. 2C shows the images of cross-sectional lesions of the aortic sinus harvested from an atherosclerosis-induced animal model to which CTS was administered, and a result of quantifying the lesion area. FIG. 2D shows a co-localization analysis result of the aortic sinus harvested from an atherosclerosis-induced animal model to which CTS was administered, after immunofluorescence staining with LC3 and SQSTM1 antibodies. FIG. 2E shows an immunofluorescence analysis result of primary peritoneal macrophages extracted from each test group, with LC3 or BODIPY, respectively. FIG. 2F shows the PLA (IP3R-VDAC1) assay images of aortic sinus sections from ApoE−/− mice treated with Veh and CTS. FIG. 2G shows the PLA (IP3R-VDAC1) assay images of primary macrophages extracted from the abdominal cavity of ApoE−/− mice treated with Veh and CTS.

FIGS. 3A-3F show that CTS reduces ER-mitochondrial contacts. FIG. 3A shows the TEM image of HUVECs treated with DMSO or CTS. The red arrows indicate the contact sites of the endoplasmic reticulum and mitochondria. FIG. 3B shows the distance between the endoplasmic reticulum and mitochondria. FIG. 3C shows the relative area of mitochondria. FIG. 3D shows the circularity of mitochondria. FIG. 3E shows a result of treating HUVECs treated or untreated with oxLDL with CTS, loading Rhod2 in the cells, live imaging the cells by confocal microscopy, and observing the extent of (Ca²⁺) release from the endoplasmic reticulum into mitochondria induced by histamine. FIG. 3F shows the PLA (IP3R-VDAC1) assay images of HUVECs treated or untreated with oxLDL, after treating with CTS, and a result of quantifying PLA dots therefrom.

FIGS. 4A-4G show a result of DARTS-LC-MS/MS analysis to identify the targets of CTS. FIG. 4A is a schematic representation of a DARTS-LC-MS/MS experiment process. M1 represents a control group, M2 represents a group treated with Protease only, and M3 represents a group treated with Protease and CTS. The PCA plot shows clustering between samples. FIG. 4B is a schematic representation showing the number of CTS target candidates present in each cell organelle. FIG. 4C and FIG. 4D show the fold change (FC) values of HYOU1 among the candidate targets whose proteolysis was stabilized by binding to CTS. FIG. 4E shows the result of in silico docking analysis for predicting the 3D structure of HYOU1 (AF-Q9Y4L1-F1). FIG. 4F is an interaction diagram of a HYOU1-CTS binding complex showing the binding site residues predicted by in silico docking analysis. FIG. 4G is a schematic representation of seven peptide fragments which are present in the ATPase domain (NBD) and the substrate binding domain (SBD) of HYOU1 and are resistant to proteolysis in the presence of CTS. ARG217 and ASN410, which are potential binding sites of CTS and HYOU1 identified by docking analysis, belong to the peptides \textcircled{c} and \textcircled{e}, respectively.

FIGS. 5A-5E show a result of investigating whether CTS and HYOU1 bind directly in vitro. FIG. 5A shows the result of DARTS assay indicating that the proteolysis of HYOU1 by Pronase is inhibited by CTS. FIG. 5B shows the result of western blot performed after DARTS analysis to estimate the binding affinity of CTS to HYOU1, and a sigmoidal curve representing the intensity of the HYOU1 band measured therefrom. FIG. 5C shows the result of transfecting HEK293 cells with wild-type MYC-HYOU1, MYC-HYOU1R217A, or MYC-HYOU1N410D vectors for 48 hours and then conducting DARTS assay using a membrane protein pool. All the vectors were labeled with MYC, and the ectopic expression pattern of HYOU1 was observed using an anti-Myc antibody. FIG. 5D shows a result of transfecting HUVECs with wild-type MYC-HYOU1, MYC-HYOU1R217A or MYC-HYOU1N410D vectors, treating the cells with CTS, and then immunofluorescence analyzing autophagosomes by labeling with the LC3 stain. FIG. 5E shows a result heating HER293 cells treated with DMSO (control), lysing the cells, conducting western blot for soluble proteins, and investigating the effect of CTS of on HYOU1 by CETSA assay.

FIGS. 6A-6F show that CTS restores autophagic damage in HUVECs induced by oxLDL. FIG. 6A shows fluorescence images taken after labeling cytosolic calcium in CTS-treated HUVECs with Fluo-4, and a graph showing cytosolic calcium level as a function of the fluorescence intensity Fluo-4. FIG. 6B shows a result of treating HUVECs with CTS, fractionating into the cytoplasm and nucleus, and then investigating TFEB expression level by western blot assay. FIG. 6C shows a result of treating HUVECs with CTS, and investigating the expression level of LC3-II and SQSTM1 24, 48, and 72 hours later by western blot assay. FIG. 6D shows the result of western blot assay indicating that the expression and accumulation of LC3-II are increased when HUVECs were treated with the lysosomal inhibitor chloroquine (CQ) along with CTS. This result suggests that CTS enhances autophagy even in the presence of the lysosomal inhibitor. FIG. 6E shows fluorescence images taken after staining CTS-treated HUVECs with Lyso Tracker Red, and a result of measuring fluorescence intensity therefrom. FIG. 6F shows the image of oxLDL visualized by Dil fluorescence after incubation of HUVECs with CTS, Dil-conjugated oxLDL, and BafA1 for 48 hours.

FIGS. 7A-7D show a result of verifying that the target of CTS is HYOU1. FIG. 7A shows a result of transfecting HUVECs with si-HYOU1 (20 nM) and then investigating the expression level of HYOU1, SQSTM1 and LC3 by western blot assay. FIG. 7B shows the result of PLA assay to identify the effect of HYOU1 knockdown on the interaction between ER (IP3R) and mitochondria (VDAC1). FIG. 7C shows fluorescence images taken after labeling cytoplasmic calcium with Fluo-4 following HYOU1 knockdown, and a graph indicating cytoplasmic calcium level as a function of the fluorescence intensity of Fluo-4. FIG. 7D shows fluorescence images taken after staining oxLDL-treated HYOU1-knockdown HUVECs with BODIPY, and fluorescence intensity measured therefrom.

FIGS. 8A-8B show that CTS has an effect of ameliorating pulmonary arterial hypertension. FIG. 8A shows PLA (IP3R-VDAC1) assay images taken after treating a pulmonary arterial hypertension (PAH) cell model, in which human pulmonary artery smooth muscle cells (PASMCs) were treated with TGF-α to induce cell contraction, with CTS, and a result of quantifying PLA dots therefrom. FIG. 8B shows a result of treating the pulmonary arterial hypertension (PAH) cell model with CTS, and then identifying the expression level of autophagy markers (p62 and LC3B-II) and a smooth muscle cell proliferation-related marker (α-SMA) by western blot assay.

FIG. 9 is a schematic representation of a novel mechanism regulating MAM and autophagy.

### DETAILED DESCRIPTION

In the present specification, the term “autophagy” refers to a mechanism related to cell survival and death that involves the constant death and renewal of cell organelles, as opposed to apoptosis or necrosis, which involves the death of the entire cell. Through this process, intracellular damage is removed, intracellular structure is reconstituted, and the intracellular supply of nutrients, energy and proteins necessary for survival is restored, allowing the cell to adapt to a stressful environment and regain homeostasis. Autophagy is an essential and evolutionarily well-conserved pathway that plays an important role in maintaining cellular function and viability in response to stress. Recently, many studies were conducted to reduce oxidative stress caused by ototoxicity using autophagy.

The present disclosure will now be described in detail.

The present disclosure relates to a pharmaceutical composition for preventing or treating autophagy disorder-related diseases, which contains cryptotanshinone (CTS) or a pharmaceutically acceptable salt thereof as an active ingredient.

In a specific exemplary embodiment, the CTS exhibits one or more characteristics selected from 1) to 4):
1) inhibition of calcium transfer from the endoplasmic reticulum to mitochondria;
2) increase of lysosomal activity;
3) increase of autophagic activity; and
4) increase of distance between the endoplasmic reticulum and mitochondria.

In a specific exemplary embodiment, the CTS binds to the endoplasmic reticulum protein HYOU1 (hypoxia-upregulated protein 1) to induce autophagy.

In the present specification, the term “autophagy disorder-related disease” means any disease caused by the occurrence of abnormal cells or proteins which are not removed by autophagy. For example, it includes a disease with defect in the maturation of autophagosomes required for autophagy.

In the present specification, the term “abnormal cell” means a cell that is morphologically or functionally significantly distinct from a normal cell. The abnormal cells include all of nutrient-depleted cells, genetically altered cells with impaired cell morphology and function, physically damaged cells, and the like.

In the present specification, the term “abnormal protein” means a protein that is morphologically or functionally significantly distinct from a normal protein. The abnormal proteins include all aggregate-prone mutant proteins and the like in which the tertiary structure is modified by amino acid modifications and the like.

The autophagy disorder-related disease may be a disease caused by the accumulation of abnormal proteins induced by reduced intracellular autophagy, or a degenerative disease.

The disease caused by the accumulation of abnormal proteins induced by reduced intracellular autophagy, or the degenerative disease may be any one selected from arteriosclerosis, pulmonary hypertension, Alzheimer's disease, Parkinson's disease, type 2 diabetes, amyotrophic lateral sclerosis, dialysis-related amyloidosis, cystic fibrosis, sickle cell anemia, Huntington's disease, Creutzfeldt-Jakob disease, Lewy body dementia, inclusion body myositis, cerebral amyloid angiopathy, traumatic brain injury, frontotemporal dementia, progressive supranuclear palsy, corticobasal degeneration, Pick's disease, and argyrophilic grain disease.

In a specific exemplary embodiment, the arteriosclerosis may be atherosclerosis.

The composition may reduce intracellular lipids by inducing autophagy.

In a specific exemplary embodiment, the pulmonary hypertension may be pulmonary arterial hypertension.

In a specific example of the present disclosure, it was confirmed that the administration of the CTS of the present disclosure to an atherosclerosis-induced animal model (ApoE−/− mice fed a high-cholesterol diet) resulted in a significant reduction in atherosclerotic plaques in the aorta and a significant reduction in the lesion area of the aorta, as compared to a control group. This suggests that the CTS of the present disclosure is effective in alleviating or ameliorating arteriosclerosis, particularly atherosclerosis.

In a specific example of the present disclosure, it was confirmed that the treatment of TGF-β-stimulated human pulmonary artery smooth muscle cells (PASMCs), i.e., a cell model of pulmonary arterial hypertension, with the CTS of the present disclosure significantly decreased endoplasmic reticulum-mitochondria interactions, significantly increased autophagy, and decreased the expression of smooth muscle cell proliferation-related markers by approximately 40%, as compared to a control group. This suggests that the CTS of the present disclosure is effective in alleviating or ameliorating pulmonary hypertension, particularly pulmonary arterial hypertension.

As used in the present disclosure, the term “prevention” refers to any action that controls or delays the autophagy disorder-related disease.

As used herein, the term “treatment” refers to reversing, alleviating the symptoms of, inhibiting the progression of, or preventing the autophagy disorder-related disease, unless stated otherwise.

The pharmaceutical composition of the present disclosure may be prepared using a pharmaceutically suitable and physiologically acceptable adjuvant in addition to the active ingredient described above, and the adjuvant may include an excipient, a disintegrant, a sweetener, a binder, a coating agent, an extender, a lubricant, a glidant, a flavoring agent, etc.

The pharmaceutical composition may be specifically formulated for administration as a pharmaceutical composition containing one or more pharmaceutically acceptable carrier in addition to the active ingredient described above.

The pharmaceutical composition may be in the form of a granule, a powder, a tablet, a coated tablet, a capsule, a suppository, a liquid, a syrup, a juice, a suspension, an emulsion, a medicinal drop, an injectable liquid, etc. For example, for formulation into a tablet or a capsule, the active ingredient may be combined with an oral, non-toxic, pharmaceutically acceptable inert carrier such as ethanol, glycerol, water, etc. Furthermore, if desired or necessary, a suitable binder, lubricant, disintegrant, a colorant or a mixture thereof may also be included in the formulation. Suitable binders include natural sugars such as starch, gelatin, glucose or beta-lactose, corn sweeteners, natural and synthetic gums such as acacia, tragacanth or sodium oleate, sodium stearate, magnesium stearate, sodium benzoate, sodium acetate, sodium chloride, etc., although not being limited thereto. The disintegrant includes starch, methyl cellulose, agar, bentonite, xanthan gum, etc., although not being limited thereto.

A pharmaceutical carrier acceptable for a composition formulated as a liquid solution may be one that is sterile and biocompatible, such as saline, sterile water, Ringer's solution, buffered saline, albumin injection solution, dextrose solution, maltodextrin solution, glycerol, ethanol, and mixtures thereof, and other common additives such as an antioxidant, a buffer, a bacteriostat, etc. may be added as desired. In addition, an injectable formulation such as an aqueous solution, a suspension, an emulsion, etc., a pill, a capsule, a granule, or a tablet can also be prepared by adding a diluent, a dispersant, a surfactant, a binder or a lubricant.

Furthermore, the composition may be formulated appropriately depending on the particular disease or ingredients, using appropriate methods known in the art as disclosed in Remington's Pharmaceutical Science, Mack Publishing Company, Easton PA.

The pharmaceutical composition of the present disclosure may be administered orally or parenterally. For parenteral administration, it may be administered by intravenous injection, subcutaneous injection, intramuscular injection, intraperitoneal injection, transdermal administration, etc.

The pharmaceutical composition of the present disclosure may provide desirable prevention, amelioration or treatment of an autophagic disorder-related disease when it contains an effective amount of CTS or a pharmaceutically acceptable salt thereof. In the present specification, the “effective amount” refers to an amount that produces a response greater than or equal to that of a negative control group, specifically an amount sufficient to ameliorate or treat an autophagy disorder-related disease. The CTS or a pharmaceutically acceptable salt thereof may be included at a concentration of 0.05 to 50 μM, specifically 0.1 to 10 μM, more specifically 0.2 to 5 μM, based on the total pharmaceutical composition. If the content of the CTS is below the lower limit, cell viability may be excellent, but the effect of ameliorating or preventing of the autophagy disorder-related disease may not be achieved as desired. And, if it exceeds the upper limit, the effect of ameliorating or preventing of the autophagy disorder-related disease may be unsatisfactory, or toxicity may occur.

The total effective amount of the pharmaceutical composition of the present disclosure may be administered to a patient as a single dose, or may be administered according to a fractionated treatment protocol in which multiple doses are administered over an extended period of time. The content of the active ingredient in the pharmaceutical composition of the present disclosure can vary depending on the severity of the disease.

Suitable dosages of the pharmaceutical composition vary depending on factors such as method of formulation, mode of administration, the age, body weight, sex, pathological condition and diet of a patient, administration time, administration route, excretion rate, and response sensitivity. An ordinarily skilled physician can readily determine and prescribe a dosage effective for the desired treatment or prevention. According to a specific exemplary embodiment, a preferred daily dosage of the pharmaceutical composition may be 0.01 to 500 mg/kg, specifically 0.1 to 100 mg/kg.

The present disclosure also provides a food composition for preventing or ameliorating of autophagy disorder-related diseases, which contains cryptotanshinone (CTS) or a sitologically acceptable salt thereof as an active ingredient.

The terms “CTS”, “autophagy”, “autophagy”, and “autophagy disorder-related disease” have already been described above and thus description thereof will be omitted here to avoid undue redundancy.

The food composition according to the present disclosure can be formulated as a powder, a granule, a tablet, a capsule, etc. using a sitologically suitable and physiologically acceptable adjuvant, and can be utilized as a functional food. The adjuvant may include an excipient, a disintegrant, a sweetener, a binder, a coating agent, an extender, a lubricant, a glidant, a flavoring agent, etc.
Furthermore, the food composition according to the present disclosure may be, for example, a beverage, an alcoholic beverage, confectionery, a diet bar, a dairy product, meat, chocolate, pizza, ramen, other noodles, a chewing gum, an ice cream, etc.

The food composition of the present disclosure may contain an ingredient commonly added in food preparation, e.g., a protein, a carbohydrate, a fat, a nutrient, a seasoning, and a flavoring agent, in addition to the CTS. Examples of the carbohydrates include common sugars such as monosaccharides, e.g., glucose, fructose, etc.; disaccharides, e.g., maltose, sucrose, oligosaccharides, etc.; and polysaccharides, e.g., dextrin, cyclodextrin, etc., as well as sugar alcohols such as xylitol, sorbitol, erythritol, etc. As the flavoring agent, a natural flavoring agent [thaumatin, stevia extract (e.g. rebaudioside A, glycyrrhizin, etc.)] or a synthetic flavoring agent (saccharin, aspartame, etc.) can be used. For example, when the food composition of the present disclosure is formulated into a beverage or a drink, it may further contain citric acid, high-fructose corn syrup, sugar, glucose, acetic acid, malic acid, fruit juice, various plant extracts, etc. in addition to the CTS of the present disclosure.

The present disclosure also provides a composition for activating autophagy in vitro, which contains cryptotanshinone (CTS) as an active ingredient.

The “CTS” and “autophagy” have already been described above and thus description thereof will be omitted here to avoid undue redundancy.

In a specific exemplary embodiment, the composition of the present disclosure can enhance autophagic activity in cells in vitro.

The cells may be cancer cells, human vascular endothelial cells (HUVECs), macrophages, or pulmonary artery smooth muscle cells.

The present disclosure also provides a composition for inhibiting endoplasmic reticulum-mitochondrial interactions in vitro, which contains cryptotanshinone (CTS) as an active ingredient.

The “CTS” has already been described above and thus description thereof will be omitted here to avoid undue redundancy.

In a specific example, the composition of the present disclosure reduced the contact of the endoplasmic reticulum and mitochondria in cells in vitro, increased the distance between the endoplasmic reticulum and mitochondria, and thereby inhibited the interaction between the cell organelles.

The cells may be cancer cells, human vascular endothelial cells (HUVECs), macrophages, or pulmonary artery smooth muscle cells.

The present disclosure will now be described in more detail with reference to examples. These examples are intended solely to illustrate the present disclosure more specifically, and it will be apparent to one of ordinary skill in the art that the scope of the present disclosure is not limited by the examples.

### EXAMPLES

### <Materials and Methods>

### Compound

Cryptotanshinone (purity≥98%, HPLC) (hereafter, referred to as ‘CTS’) was purchased from Sigma-Aldrich (St. Louis, MO, USA).

### Cell Culturing

HUVECs (4-9 passages) were cultured in EBM-2 basal medium (Lonza, CC-3156) containing supplements (Lonza, CC-4176). In addition, HEK293, HepG2, HeLa and RAW264.7 cells were cultured in DMEM supplemented with 10% FBS and 1% antibiotics, respectively. All the cells were cultured in a humidity-controlled incubator under the condition of 5% CO₂, 37° C. and pH 7.4.

### MTT Assay

HUVECs were seeded into a 96-well plate (3×10³ cells/well) and incubated overnight. The cells were then treated with CTS at various concentrations and incubated for 24-72 hours. Then, after adding MTT solution (3-(4,5-dimethylthiazol-2-yl)-2,5-diphenyltetrazolium bromide; Sigma-Aldrich) to each well at a concentration of 0.4 mg/mL (final concentration), the cells were left for 3 hours. Then, after completely removing the medium containing the MTT solution, MTT formazan product in each well was dissolved in DMSO, and absorbance was measured at a wavelength of 595 nm with a microplate reader.

### Immunoblotting Assay

The cell culture was harvested and the expression of proteins was confirmed by immunoblot assay.

Specifically, the cells were lysed using a 2×SDS lysis buffer (0.12 M Tris-Cl, pH 6.8, 3.3% SDS, 10% glycerol, 3.1% DTT) to obtain a cell lysate. Proteins were separated by SDS-polyacrylamide gel electrophoresis (SDS-PAGE; resolving buffer 1.5 M Tris-Cl, pH 8.8, stacking buffer 0.5 M Tris-Cl, pH 6.8), and the separated proteins were transferred to a polyvinylidene difluoride (PVDF) membrane (BioRad, USA). The membrane to which the proteins were transferred was incubated with primary antibodies [anti-SQSTM1/SQSTM1 (5114s, CST), anti-LC3B (2775s, CST), anti-Myc, anti-TFEB, anti-β-actin (ab6276, Abcam), and anti-β-tubulin (Abcam)] diluted in 3% (w/v) skim milk or 5% (w/v) BSA overnight at 4° C. The membrane was then washed with a TBST buffer (10 mM Tris-HCl, 150 mM NaCl, 0.05% Tween 20, pH 7.4) and incubated with appropriate secondary antibodies (1:3000 v/v, GE Healthcare) for 1 hour at 25° C. Subsequently, the proteins were detected using an enhanced chemiluminescence substrate (BioRad) according to the manufacturer's instructions, and immunoblot bands were visualized using the ChemiDoc XRS+ imaging system (BioRad). B-Actin and B-tubulin were used as internal standards.

### Transfection

For gene knockdown study, HUVECs were transfected with 20 nM siRNA (Dharmacon™, Lafayette, CO, USA, L-003678-00-0005) for 24-48 hours using the Lipofectamine RNAiMAX transfection reagent (Invitrogen). HYOU1 ORF clones with a Myc-DDK tag (OriGene, Rockville, MD, USA, RC214178) were used for production of the HYOU1 mutant vector. The cells were transfected with a plasmid (HYOU1 human-tagged ORF clones) according to the manufacturer's instructions using the Lipofectamine LTX reagent (Invitrogen). For transfection with the mRFP-GFP-LC3B vector, HUVECs were transfected with the plasmid (mRFP-GFP-LC3B) for 24 hours using the Lipofectamine LTX transfection reagent (Invitrogen). Subsequently, the nuclei of the cells were stained with Hoechst 33432 and the cells were fixed with 4% formaldehyde. Images were then obtained using an LSM880 confocal microscope at 400× magnification and quantified by counting red and green puncta.

Transfection with SPLICS Vector

HUVEC cells were cultured overnight in a 12-well plate with a coverslip at a density of 5.0×10⁵ cells/well. The cells were transfected with the SPLICS (ER-Mito) vector using the Lipofectamin LTX reagent. Images were then captured in different z-planes using a confocal microscope (LSM980). For quantification, the area of GFP fluorescence relative to the total cell area (%) was measured with the Image J software.

### PLA (Proximity Ligation Assay)

HUVECs were cultured overnight at a density of 1.5×10⁴ cells/well in a chamber-type cell culture slide (8-well) and treated with 50 μg/mL oxLDL for 24 hours or with 5 μM CTS for 4 hours. Subsequently, PLA was conducted using the Duolink in-situ red kit (Sigma-Aldrich, St. Louis, MO) according to the manufacturer's instructions. Specifically, the cells were fixed with 4% PFA for 10 minutes and permeabilized with 0.2% Triton X-100 for 20 minutes at room temperature. The cells were then blocked for 1 hour with a blocking solution and incubated with primary antibodies overnight at 4° C. Then, the cells were treated with PLA probes (anti-rabbit PLUS and anti-mouse MINUS) and incubated at 37° C. for 1 hour. The cells were incubated with a ligase for 30 minutes and then incubated with a DNA polymerase for 100 minutes at 37° C. The sample was then mounted using a mounting medium containing DAPI. Subsequently, PLA signals were observed using a Zeiss LSM 980 confocal microscope and quantified by counting the number of PLA dots.

### Drug Affinity Responsive Target Stability (DARTS) Assay

HepG2 or HUVECs (1×10⁷ cells) were lysed by homogenization in PBS buffer containing a protease/phosphatase inhibitor cocktail (½ tablet, 25 mL; Pierce), and the concentration of total proteins was determined by BCA assay. All steps were performed on ice to avoid premature protein degradation. The cell lysate (final protein concentration: 1 mg/mL, 100 μL) was aliquoted into a 1.5-mL tube and incubated with 0.01 to 10 μM CTS (in 1 μL DMSO) for 3 hours at 4° C. with gentle stirring. Pronase (non-specific protease) was then added (final concentration: 1 μg/mL) and reaction was performed for 10 minutes. After adding a 6×SDS buffer, the Pronase reaction was stopped by heating for 7 minutes.

### CETSA (Cellular Thermal Shift Assay)

After harvesting the cells using a trypsin-EDTA solution, the cell suspension (3×10⁷ cells/15 mL) was dispensed into a conical tube. Then, CTS (20 μM) was added, and DMSO was used as a control group. The cells were incubated for 1 hour in a 5% CO₂ incubator at 37° C. with gentle agitation. After centrifugation, the pellet was washed with PBS and suspended in 1 mL of PBS (with protease inhibitor). The cell suspension was dispensed into PCR tubes (˜100 μL per tube) heated at 40-64° C. for 3 minutes, and then cooled at 25° C. for 3 minutes. After centrifuging the tube to obtain pellets, the supernatant was discarded. The cell pellets were resuspended in 0.4% NP-40 (in PBS) supplemented with a protease inhibitor to facilitate the dissolution of hydrophobic proteins. For cell lysis, the cell suspension was repeatedly frozen and thawed in liquid nitrogen twice. The cell lysate was centrifuged at 20,000 g and 4° C. for 20 minutes, and the supernatant (soluble proteins) was analyzed by western blot.

### Immunocytochemistry (ICC) Assay

HUVEC cells were cultured overnight in a 12-well plate with a coverslip at a density of 5.0×10⁵ cells/well. The cells were fixed with 4% formaldehyde and permeabilized with 0.2% Triton X-100. After blocking with 1-3% BSA, the cells were incubated with primary antibodies overnight at 4° C. Subsequently, after adding a fluorescent secondary antibody conjugate (Alexa Fluor 488 or 594), the cells were at 37° C. for 1 hour. The cells were washed three times with PBS and images were obtained using an LSM980 confocal microscope.

### Calcium Assay

To measure calcium content in mitochondria or cytosol, HUVECs were cultured overnight at a density of 1.5×10⁴ cells/well in a chamber slide (8-well) and incubated with Rhod-2/AM (2 μM) (Invitrogen, R1244) or Fluo-4/AM (1 μM) (Invitrogen, F14201), respectively, for 30 minutes at room temperature. The cells were then incubated in a calcium-free Krebs-Ringer-HEPES buffer (pH 7.4) additionally for 30 minutes for de-esterification of the dye. Images were captured in real time using an LSM980 confocal microscope (Zeiss, Oberkochen, Germany).

### Fluorescence Staining

For Lysotracker (Invitrogen, L7528) staining, HUVECs were cultured overnight in a 12-well plate with a coverslip at a density of 5.0×10⁴ cells/well and then treated with CTS (5 μM) for 48 hours. The cells were then fixed in 4% formaldehyde and washed three times with PBS. The cells were then incubated with 100 nM Lysotracker for 30 minutes and the nuclei were stained with Hoechst. For BODIPY (Invitrogen, D3922) staining, HUVECs were cultured overnight in a 12-well plate with a coverslip at a density of 5.0×10⁴ cells/well and then treated with CTS (5 μM) for 48 hours. The cells were then fixed in 4% formaldehyde and washed three times with PBS. The cells were then incubated with the BODIPY fluorescence reagent for 30 minutes and the nuclei were stained with Hoechst. Images were obtained using an LSM980 confocal microscope at 400× magnification. All fluorescence signal intensities were quantified using the Image J software.

### Foam Cell Formation

RAW264.7 macrophages were seeded in a 12-well plate at a density of 10×10⁴ cells/well and cultured overnight. The cells were then treated with 50 μg/mL oxLDL (Invitrogen) for 48 hours. The cells were fixed and stained with Oil Red O to determine whether the normal RAW264.7 cells were changed to foam cells. The cells were examined under a light microscope and images were obtained.

### In Silico Docking Study

Molecular docking analysis was performed using the Discovery Studio 2018 software (Accelrys, San Diego, CA, USA). Specifically, after securing the three-dimensional structure of the HYOU1 protein and ligands, experiment was proceeded in the order of designation of the binding site considering the protein structure, etc. followed by docking of the ligands using the CDOCKER program.

First, after obtaining the structure of the HYOU1 protein from the Alphafold database, the chemical structure of the ligands was investigated using the Discovery Studio 3.5 program from BIOVIA. Then, binding sites were designated in the receptor cavities using the CDOCKER (grid-based molecular docking method using CHARMm force field) module, which is a program that finds and provides the most favorable binding between small molecules and the active sites of proteins. The ligands were docked into the binding sites of the protein to produce the 10 best hit poses, and then binding energy (CDOCKER energy) was calculated.

### Animal Experiment

Mouse experiments were performed in accordance with the ethical guidelines approved by the Institutional Animal Care and Use Committee of Yonsei University (IACUC-A-202208-1519-01). ApoE−/− mice were provided by Dr. Goo Taeg Oh (Ewha Womans University, Korea) and were housed in a pathogen-free system with a 12-hour light-dark cycle with free access to water and food. The mice were fed a high-cholesterol diet from 6 weeks after birth until the end of the experiment. At the end of the experiment, the mice were anesthetized with Avertin (2,2,2-tribromoethanol) and perfused with PBS through the left ventricle.

### Analysis of Atherosclerotic Lesions in Tissue

The outer membrane fat of the aorta isolated from the sacrificed animals was removed, dissected longitudinally, and fixed on a white silicone plate. After overnight fixation with 4% PFA, aortas were stained overnight with Oil Red O and washed with PBS. The heart and liver were then harvested and fixed overnight in 4% PFA and then placed in a 30% sucrose solution. After preparing tissues and preserving through cryopreservation (OCT) or paraffin embedding, sections were prepared for histologic analysis. For analysis of atherosclerotic lesions, the bottom of the heart, including the proximal aorta, was embedded in OCT medium, stained using the Oil Red O stain, and then quantified using the Image J software.

### Immunofluorescence Staining of Tissue Sections

Tissue sections were embedded in paraffin blocks for fluorescence immunohistochemistry. Paraffin-embedded tissue slides were immersed in xylene to remove paraffin and rehydrated in ethanol. Then, the antigens were recovered using a citrate buffer (pH 6.0). After blocking sections with 5% BSA for 30 minutes, they were incubated with the primary anti-LC3 antibody (ab48394) in a culture buffer (3% BSA containing 0.2% Triton X-100 in PBS) overnight at 4° C. After washing three times with PBS, the sections were incubated with secondary antibodies having Alexa Fluor 488 and 594 tags for 1 hour. After staining the nuclei with DAPI, the result was analyzed by confocal microscopy.

### Isolation of Peritoneal Macrophages

The accumulation of macrophages was induced by injecting 3 mL of a 3% thioglycolate medium into each mouse 4 days before the end of the animal experiment. After sacrifice, a small incision was made in the abdomen of each mouse and approximately 5 mL of cold harvest medium (DMEM) was injected into the peritoneum. Using the same syringe, peritoneal fluid was aspirated from the peritoneum. The extracted peritoneal fluid was centrifuged to obtain cell pellets, which were then incubated with an RBC lysis buffer on ice to remove red blood cells. The collected macrophages were then inoculated onto a plate. After a few hours, the plate was washed gently with warm medium after removing unattached cells.

### Culturing of PASMCs

Human pulmonary artery smooth muscle cells (PASMCs; 4-8 passages) were cultured in an SmGM2 basal medium (Lonza, CC-3181) containing 5% FBS, supplements (Lonza, CC-4149) and 1% antibiotics.

### PLA (Proximity Ligation Assay)

The cells were cultured in a chamber-type cell culture slide. When they reached 70% confluence, the medium was replaced with a medium containing 0.5% FBS and the cells were incubated for 4 hours to inhibit cell proliferation. The cells were then treated with DMSO and CTS (5 μM) while simultaneously stimulating with TGF-β (5 ng/mL) for 24 hours. After performing PLA assay for IP3R and VDAC1, PLA signals were observed using a Zeiss LSM 980 confocal microscope and quantified by counting the number of PLA dots.

### Identification of Autophagy and Smooth Muscle Cell Proliferation Markers

The cells were cultured in a chamber-type cell culture slide. When they reached 70% confluence, the medium was replaced with a medium containing 0.5% FBS and the cells were incubated for 4 hours to inhibit cell proliferation. The cells were then treated with DMSO and CTS (5 μM) while simultaneously stimulating with TGF-β (5 ng/ml) for 24 hours. The level of p62, LC3 and α-SMA was confirmed by western blot.

### Statistical Analysis

All data were collected using GraphPad Prism (Windows ver. 9; GraphPad Software, Inc., San Diego, CA) and expressed as mean±SEM. Statistical analysis was performed by unpaired two-tailed Student t-test. P-values<0.05 were considered statistically significant (*p<0.05, **p<0.01, ***p<0.001).

### <Experimental Results>

### OxLDL Disrupts Mitochondrial Homeostasis.

Endothelial cell dysfunction caused by oxLDL is one of the main causes of atherosclerosis.

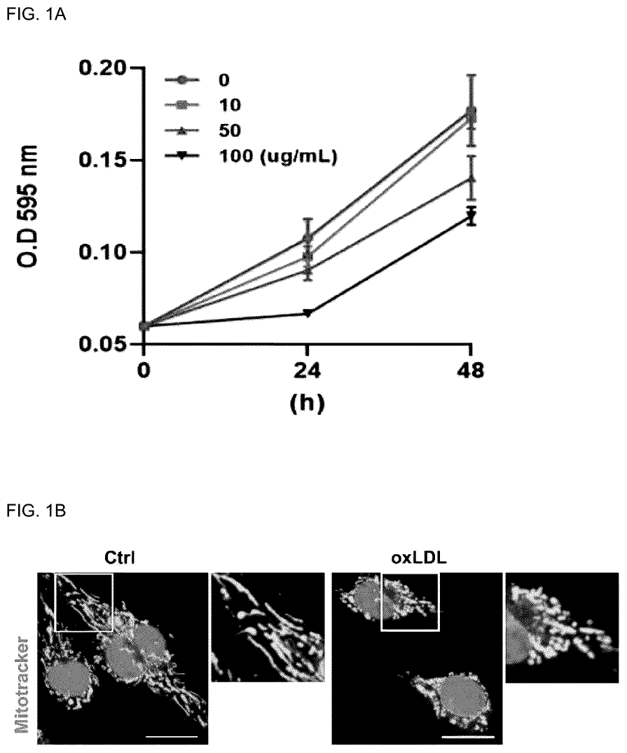

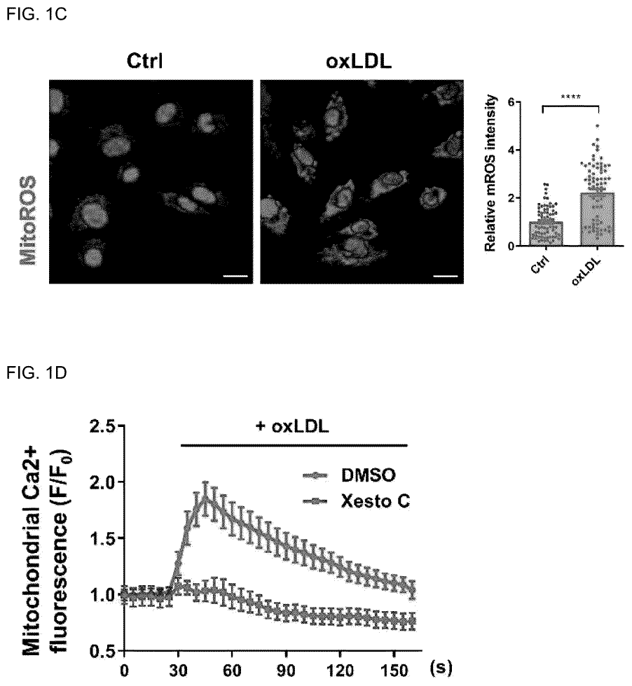

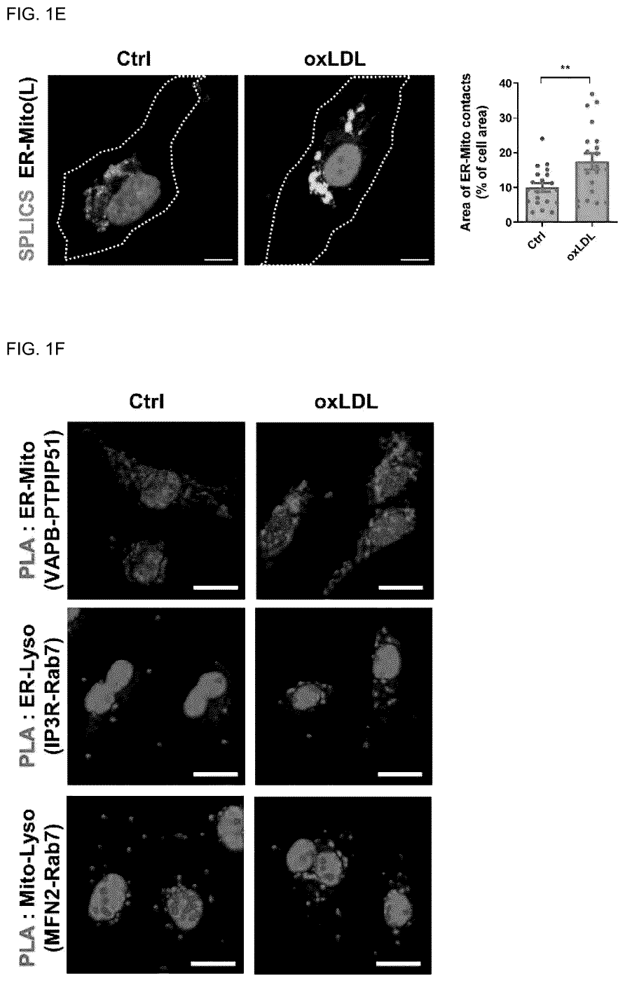

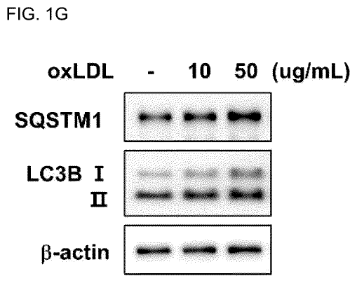

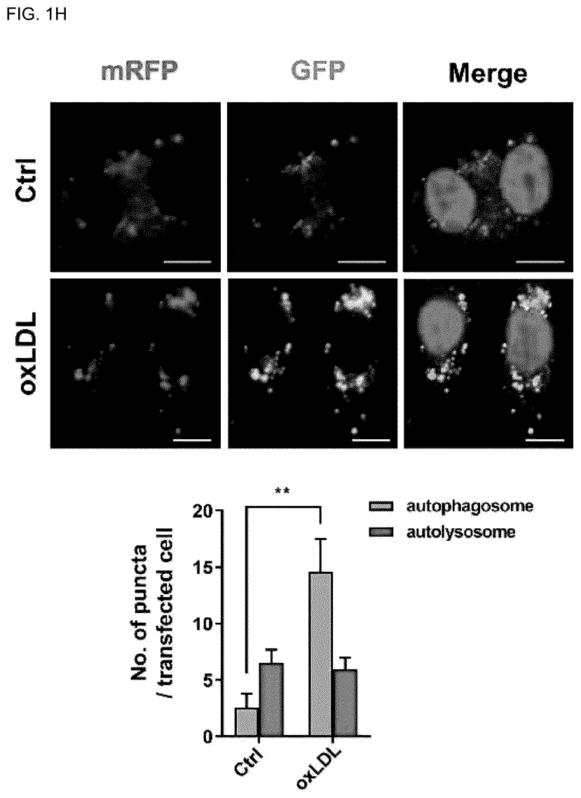

First, the effect of oxLDL on the proliferation of HUVECs was evaluated by performing MTT assay to investigate the physiological effects of oxLDL. Specifically, HUVEC cells were treated with 0-100 μg/mL of oxLDL and MTT assay was performed 24 and 48 hours later, respectively. As a result, cell proliferation was inhibited (FIG. 1A).

Furthermore, the analysis of mitochondrial morphology revealed that the HUVECs treated with oxLDL (50 μg/mL) exhibited abnormal mitochondrial morphology, such as swelling and fragmentation, as compared to the control group (FIG. 1B).

It was also found that oxLDL (50 μg/mL) significantly increased the production of mitochondrial reactive oxygen species (mitoROS) (FIG. 1C). This means that oxLDL induces oxidative stress in HUVECs.

In addition, it was investigated whether intracellular calcium was released from the ER and imported into the mitochondria in response to oxLDL. Specifically, it was confirmed that, when the HUVECs were treated with oxLDL along with the Rhod-2 dye, which is a marker of mitochondrial calcium, mitochondrial calcium level was increased. In contrast, when HUVECs were pretreated with xestospongin C, which is an IP3R antagonist, and then treated with oxLDL, there was no increase in the mitochondrial calcium level (FIG. 1D). This means that the oxLDL-induced increase in mitochondrial calcium is derived from the endoplasmic reticulum (ER).

Next, the change in endoplasmic reticulum-mitochondrial interactions induced by oxLDL was investigated.

To this end, SPLICS (ER-mitochondria split-GFP) assay was performed to reveal the interactions between cell organelles in oxLDL-treated HUVECs. SPLICS is an efficient tool for imaging the proximity of cell organelles in a cell based on the principle that split-GFP fluorescence is expressed when two cell organelles are in close proximity. When two cell organelles move closer together, fluorescence is emitted at the junction due to complementation of GFP.

First, HUVECs were transfected with the SPLICS vector to observe the changes in the distance between ER and mitochondria caused by oxLDL treatment. The HUVECs were then treated with oxLDL (50 μg/mL) and the area (%) of GFP fluorescence relative to the total area per cell was measured. FIG. 1E shows that, 24 hours after oxLDL treatment, the fluorescence signal of OMM-GFPβ1-10 connected to the long linker ER-GFPβ11 was increased. This result suggests that oxLDL increases endoplasmic reticulum-mitochondrial interactions and disrupts mitochondrial homeostasis, leading to cellular dysfunction.

To determine whether oxLDL also affects the interactions of other cell organelles, PLA was performed using markers for each cell organelle. The result showed that treatment with oxLDL (50 μg/mL) significantly increased ER-mitochondria contacts, but did not significantly affect mitochondria-lysosome contacts in comparison (FIG. 1F).

Autophagy is a key mechanism for maintaining intracellular homeostasis. The effect of oxLDL on intracellular autophagic activity was investigated. To this end, HUVECs were treated with oxLDL (10, 50 g/mL) and then the expression level of SQSTM1 and LC3 proteins, which are autophagy-related markers, was investigated. As a result, it was found that oxLDL increased the expression and accumulation of SQSTM1 and LC3 proteins in a dose-dependent manner (FIG. 1G). This means that oxLDL not only disrupts mitochondrial homeostasis, but also impairs the autophagic activity of cells.

In addition, to monitor autophagy flux, HUVECs were transfected with the mRFP-GFP-LC3 vector, treated with oxLDL (50 μg/mL), and then observed by fluorescence confocal microscopy (FIG. 1H). The result showed that the oxLDL treatment significantly increased autophagosomes in the cells. This means that oxLDL not only disrupts mitochondrial homeostasis, but also impairs the autophagic activity of cells.

### CTS Ameliorates Atherosclerosis by Inducing Autophagy in ApoE Knockout Mice.

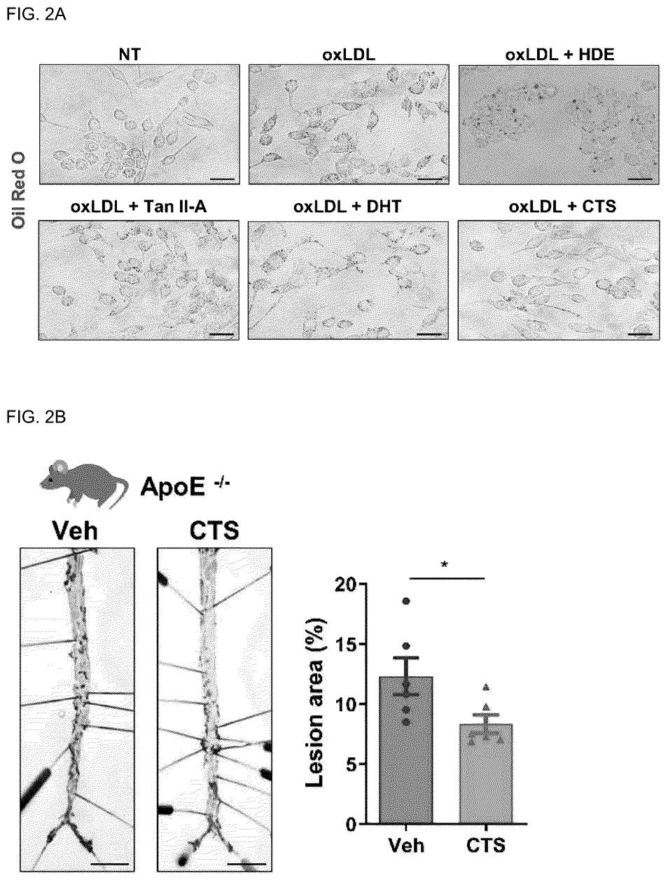

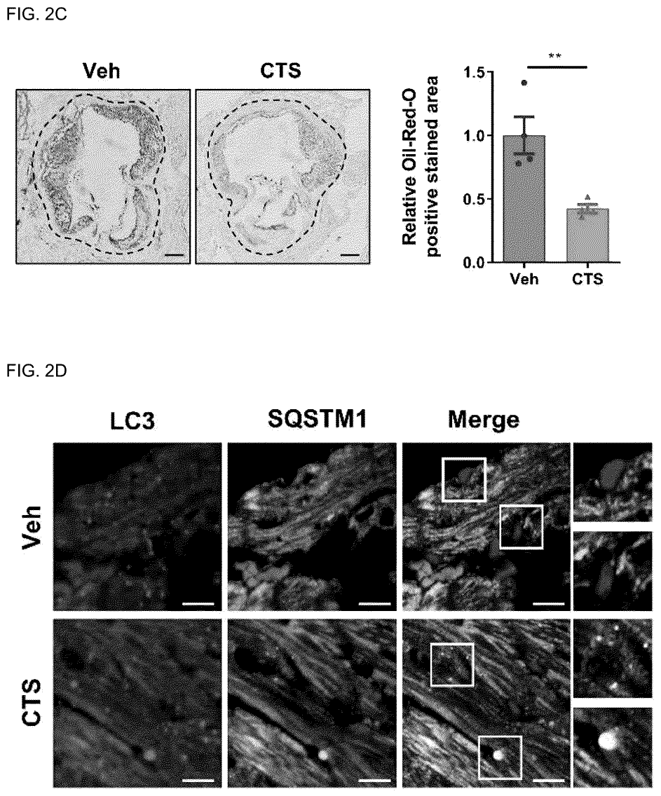

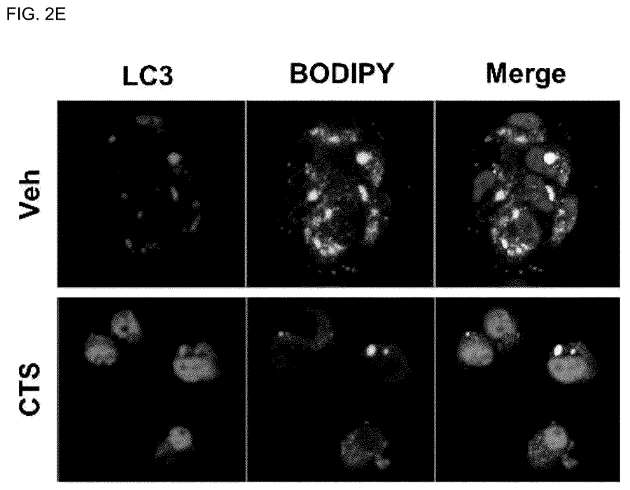

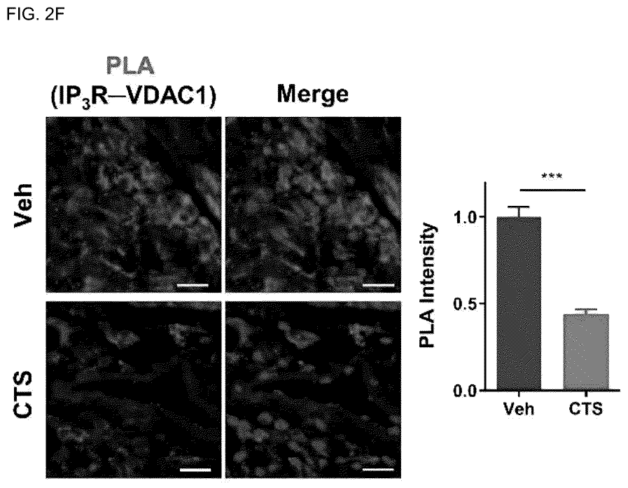

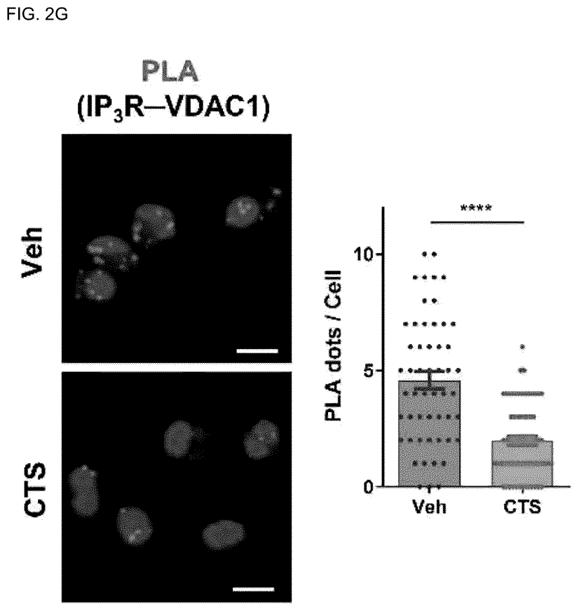

First, RAW264.7 macrophages stimulated with oxLDL were treated with HDE (danshen extract; positive control group), tanshinone II-A (T II-A), dihydrotanshinone (DHT), or cryptotanshinone (CTS), respectively, and the formation of foam cells was confirmed by staining with Oil Red O. The result showed that foam cells were significantly increased in the oxLDL treatment group as compared to a normal group (NT), while the foam cell formation was significantly inhibited in the oxLDL+CTS treatment group as compared to the oxLDL, oxLDL+T II-A and oxLDL+DHT treatment groups (FIG. 2A).

An atherosclerosis-induced animal model (ApoE−/− mice fed a high-cholesterol diet) was intraperitoneally injected with CTS (20 mg/kg) every 2 days for 8 weeks. The CTS administration did not affect the body weight of the animal model, indicating low toxicity. In addition, the CTS treatment showed reduced total cholesterol and triglyceride contents in the blood and significantly reduced atherosclerotic plaque formation in the aorta as compared to the control group (FIG. 2B). Furthermore, after cross sectioning of the proximal aorta of the cardiac tissue followed by staining of lipids with Oil Red O, a significant reduction in the lesion area was observed (FIG. 2C).

To assess the association between autophagy and atherosclerosis, the co-localization of LC3 and SQSTM1 in aortic sinus sections was compared. The result showed higher fluorescence signal intensity and increased co-localization of LC3 and SQSTM1 in the CTS group as compared to the control group (FIG. 2D). The increased co-localization of LC3 and SQSTM1 in the aortic sinus sections of the CTS group suggests increased autophagy flux. The improved autophagy can help reduce inflammation and cellular stress through better cleanup of damaged cell organelles and proteins.

Furthermore, as a result of extracting mouse primary peritoneal macrophages from each animal model and analyzing LC3 and lipid levels, it was found that, whereas the control group showed decreased LC3 and increased lipid content as compared to the normal group due to the induction of atherosclerosis, the decreased LC3 and increased lipid content were reversed in the CTS treatment group (FIG. 2E).

In addition, it was investigated whether the MAM-regulating activity of CTS is also manifested in vivo. PLA (IP3R-VDAC1) assay of aortic sinus sections from ApoE−/− mice treated with a vehicle (Veh) and CTS showed that the CTS treatment significantly reduced PLA (IP3R-VDAC1) dots in the cardiac tissue as compared to the vehicle (Veh) (FIG. 2F). Furthermore, as a result of the PLA (IP3R-VDAC1) assay for the peritoneal macrophages from the ApoE−/− mice treated with the vehicle (Veh) and CTS, it was confirmed that the CTS treatment significantly reduced PLA (IP3R-VDAC1) dots in the mouse peritoneal macrophages as compared to the vehicle (Veh) (FIG. 2G).

This result indicates that the CTS administration reduces the interaction between IP3R and VDAC1 in the heart tissue and macrophages of the atherosclerosis-induced animal model, suggesting that CTS may restore mitochondrial function and calcium homeostasis by alleviating the aberrant role of MAMs associated with atherosclerosis.

It also suggests that CTS increases autophagy in tissues undergoing atherosclerosis and may mitigate atherosclerosis induced by a high-cholesterol diet.

### CTS Inhibits Initial ER-Mitochondrial Interactions In Vitro.

It was investigated whether CTS inhibits ER-mitochondrial contacts.

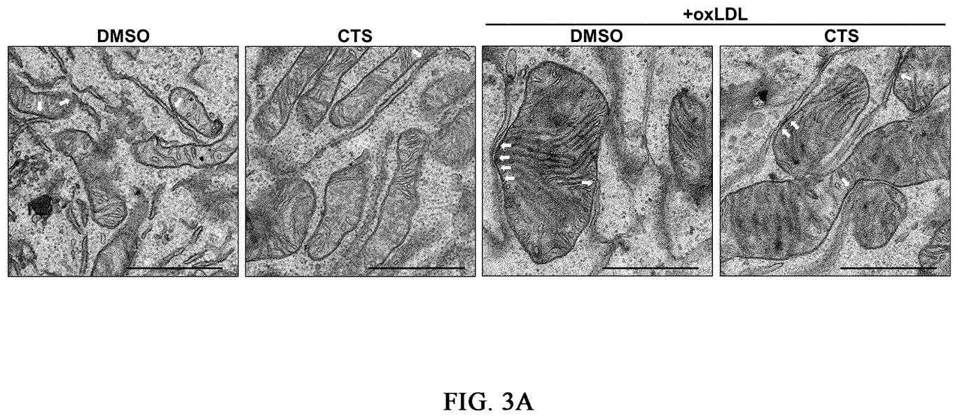

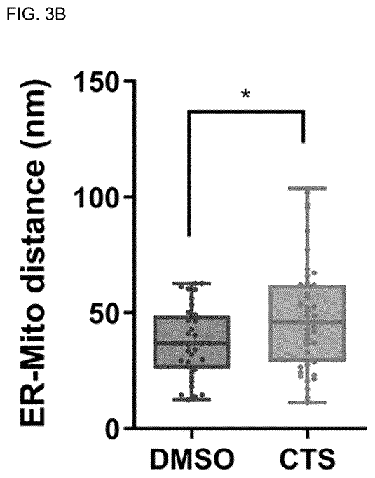

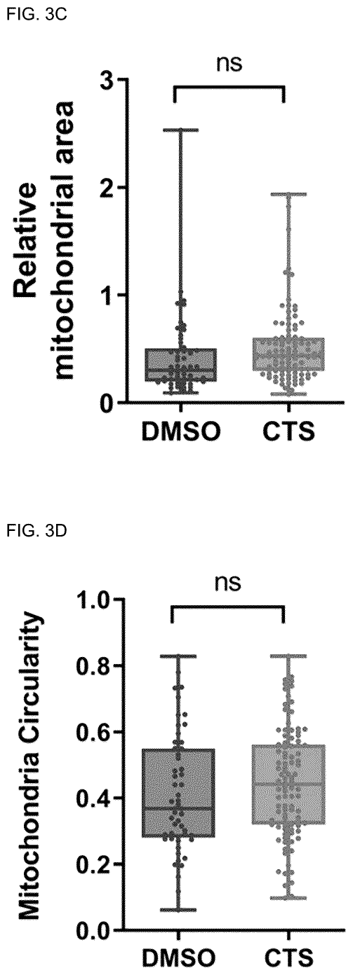

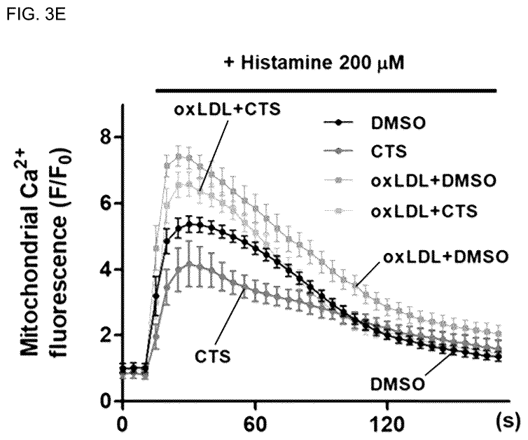

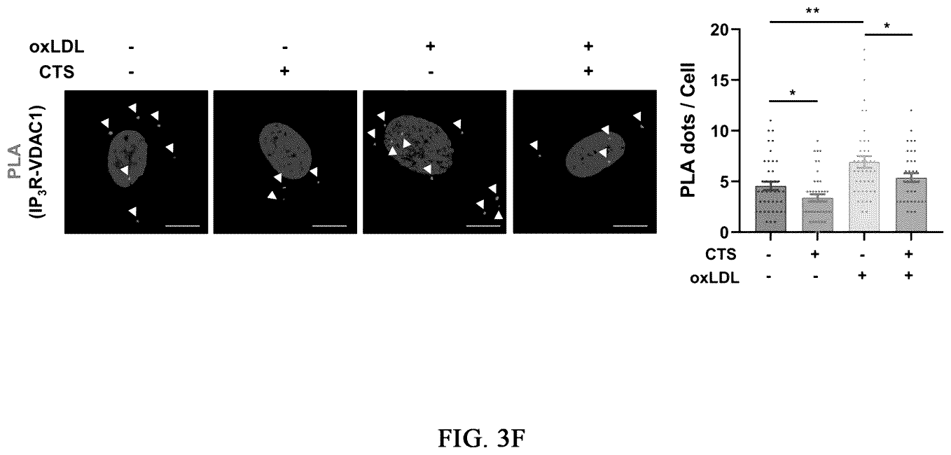

First, HUVECs were imaged by transmission electron microscopy (TEM) to visualize the ER and mitochondria, and the ER-mitochondrial distance was observed. The number of dots closer than 40 nm was 61% in the control group and 37% in the CTS-treated group (5 μM), which was significantly reduced by the CTS treatment (FIG. 3A). That is, the distance between ER-mitochondria was found to be significantly increased by the CTS treatment (FIG. 3B). This result suggests that CTS effectively inhibits abnormal interactions between cell organelles. Increased contact between the ER and mitochondria can lead to cellular stress and dysfunction. CTS can help restore cellular homeostasis and prevent adverse effects associated with increased ER-mitochondrial interactions. On the other hand, mitochondrial area and circularity were found to be unaffected by the CTS treatment (FIG. 3C and FIG. 3D). This result suggests that CTS can selectively modulate interactions between cell organelles without compromising mitochondrial integrity.

Furthermore, the PLA assay result showed that the oxLDL treatment increased the number of PLA (IP3R-VDAC1) dots, whereas treatment with oxLDL and CTS together decreased the number of PLA (IP3R-VDAC1) dots (FIG. 3F).

Furthermore, in order to investigate whether change in the physical coupling between the ER and mitochondria affects communications between cell organelles, Ca²⁺ transport from the ER to mitochondria was examined. For this, HUVECs were treated with CTS to load Rhod2 into the cells, and live imaged by confocal microscopy. Then, after inducing calcium (Ca²⁺) release from the endoplasmic reticulum with histamine, the extent of influx into mitochondria was observed. The result showed that, whereas mitochondrial calcium intensity was increased in the oxLDL-treated cells as compared to the control group, the histamine-induced calcium influx into mitochondria was decreased in the CTS-treated cells (FIG. 3E). This suggests that CTS reduced ER-mitochondrial contacts, which in turn attenuated calcium transport from the ER to the mitochondria. This means that CTS reduces ER-mitochondrial contacts, which were increased by oxLDL.

### Identification of Targets of CTS

The molecular targets of CTS associated with autophagy was identified by the DARTS-LC-MS/MS method. DARTS-LC-MS/MS (drug affinity responsive target stability (DARTS)-LC-MS/MS) is an efficient method for the identification of target proteins from label-free small molecules.

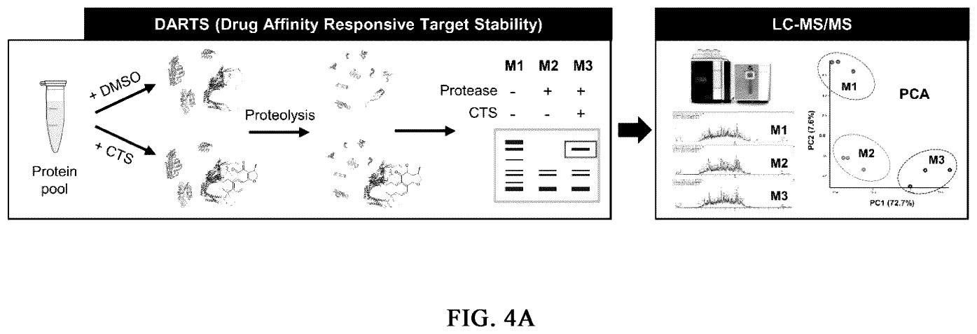

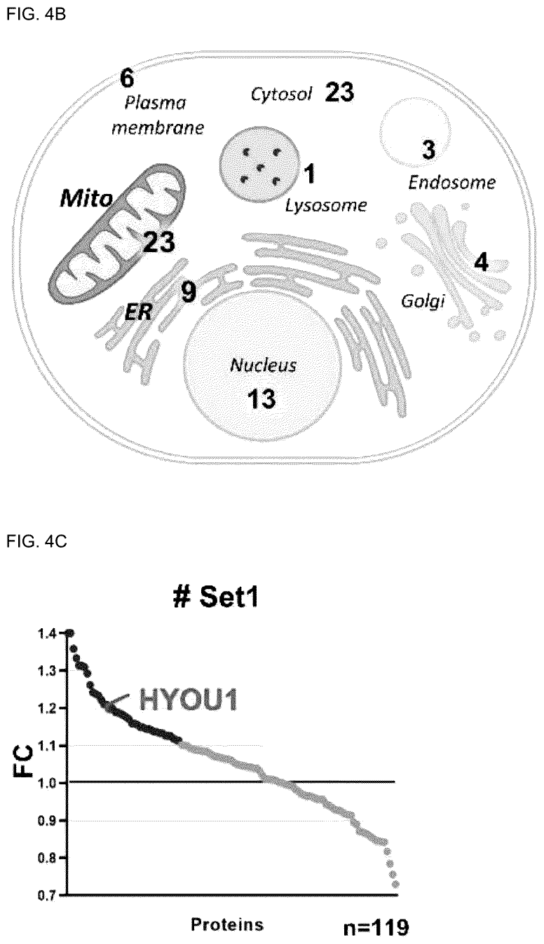

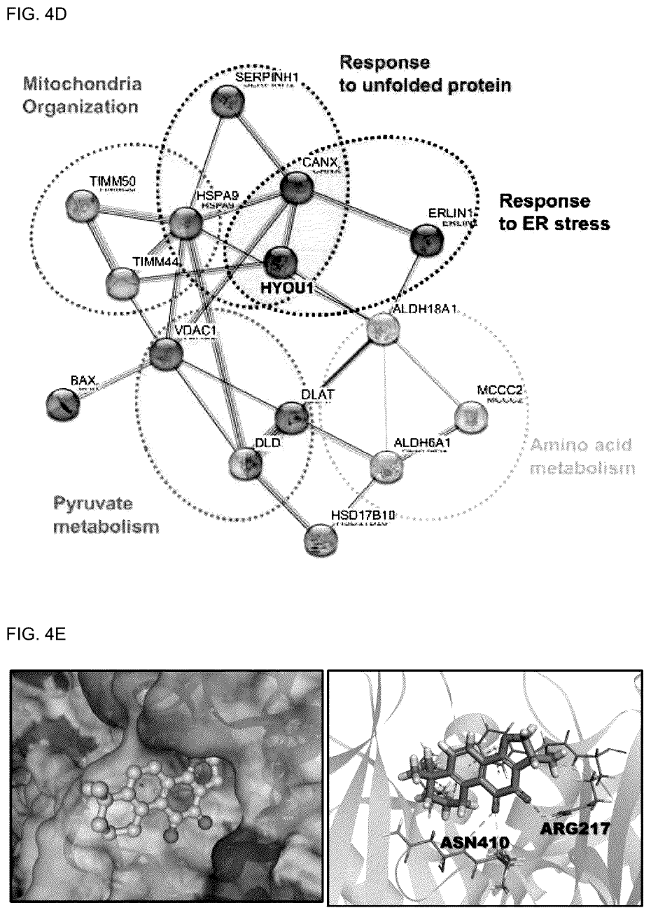

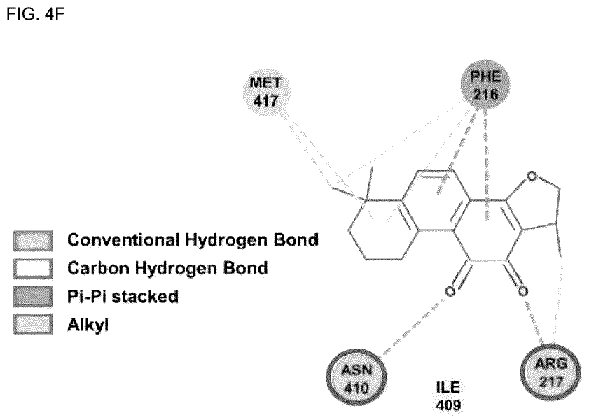

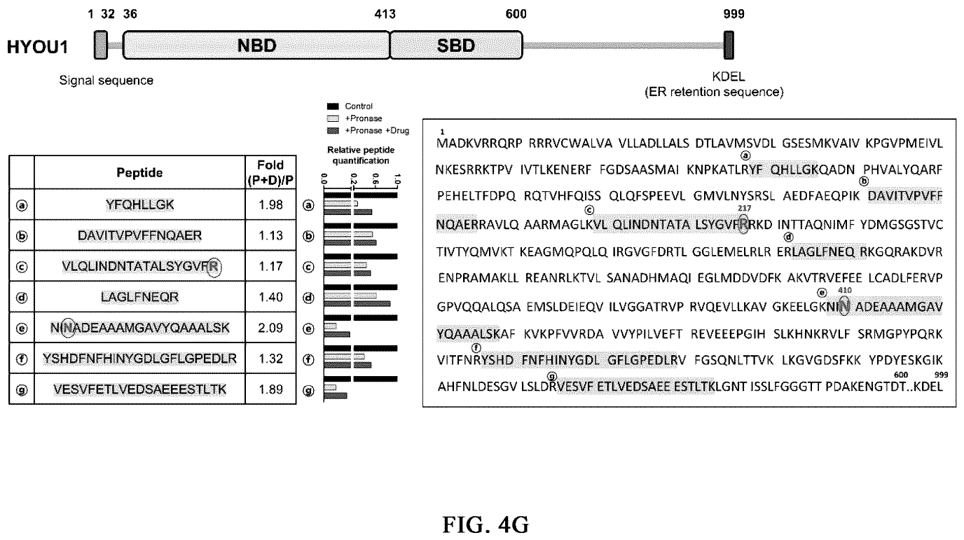

Specifically, membrane proteins were fractionated from a HepG2 cell lysate, subjected to DARTS, and analyzed by LC-MS/MS (FIG. 4A). They were clustered by principal component analysis (PCA) for three groups (M1: control group, M2: Pronase alone, M3: Pronase and CTS). The PCA result showed clear distinction between the control, Pronase alone group, and the CTS treatment group. This indicates that the treatment with CTS results in significant changes in the protein landscape. In total, about 850 proteins were detected by the above analysis. Among them, the proteins that showed at least 10% resistance to proteolysis were selected.

It was found that the selected target candidates were predominantly located in the ER (23) or mitochondria (9) among the several cell organelles (FIG. 4B).

Among the candidate targets that bind to CTS and are stabilized against proteolysis, the ER-resident chaperone HYOU1 was selected as the final target candidate with high sequence coverage while exhibiting a high level of protective activity. HYOU1 was also found to be the only common protein in the DARTS-LC-MS/MS performed additionally for a different set of protease concentration assays. FIG. 4C shows a heatmap of the top candidates that showed high levels of protection against proteolysis in combination with CTS. In addition, the gene ontology of the target candidates was examined through STRING (FIG. 4D), and a list of proteins belonging to specific categories (pyruvate metabolism, response to unfolded proteins, alpha-amino acid metabolism, response to ER stress, and mitochondrial organization) was obtained.

Next, the potential binding site of CTS was investigated by in silico docking analysis using the Alphafold 3D predicted structure of HYOU1 (AF-Q9Y4L1-F1) (FIG. 4E). The result confirmed the possibility that CTS may bind to a pocket located in the ATPase domain of HYOU1. In particular, it was predicted that ARG217 and ASN410 of HYOU1 could interact with the carbonyl group of CTS via hydrogen bonding (FIG. 4F).

Furthermore, a total of 16 peptide fragments corresponding to HYOU1 were detected by LC-MS/MS analysis. Among them, seven peptides (C to) located in the ATPase-binding domain (NBD) and the substrate-binding domain (SBD) of HYOU1 exhibited resistance to proteolysis in the presence of CTS (FIG. 4G). ARG217 and ASN410 were predicted as binding sites for CTS and HYOU1, and were found to be in the and peptide sequences of the NBD, respectively.

### HYOU1 is a Target Protein of CTS.

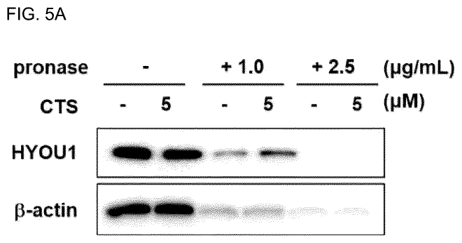

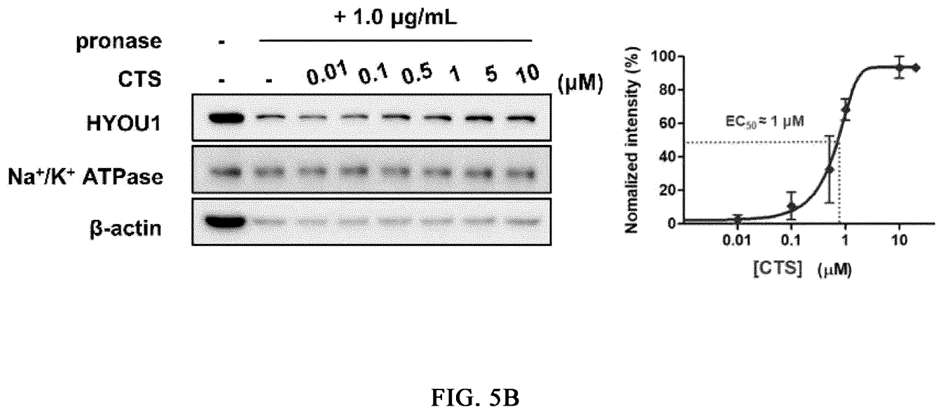

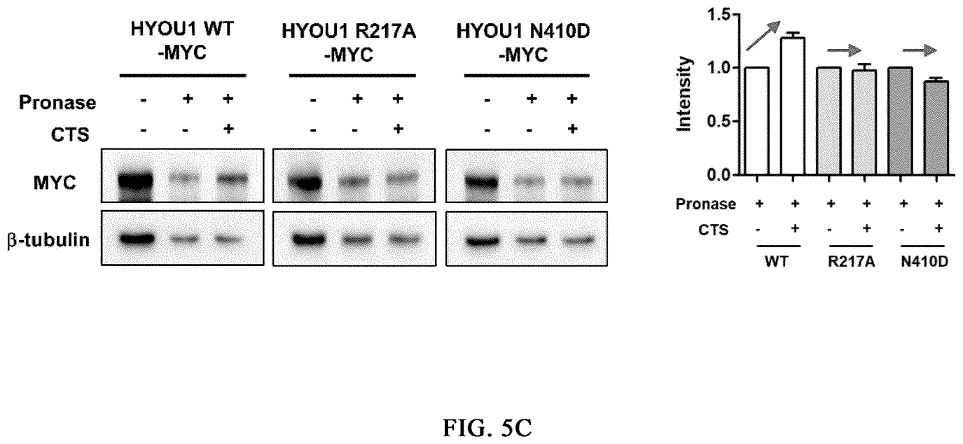

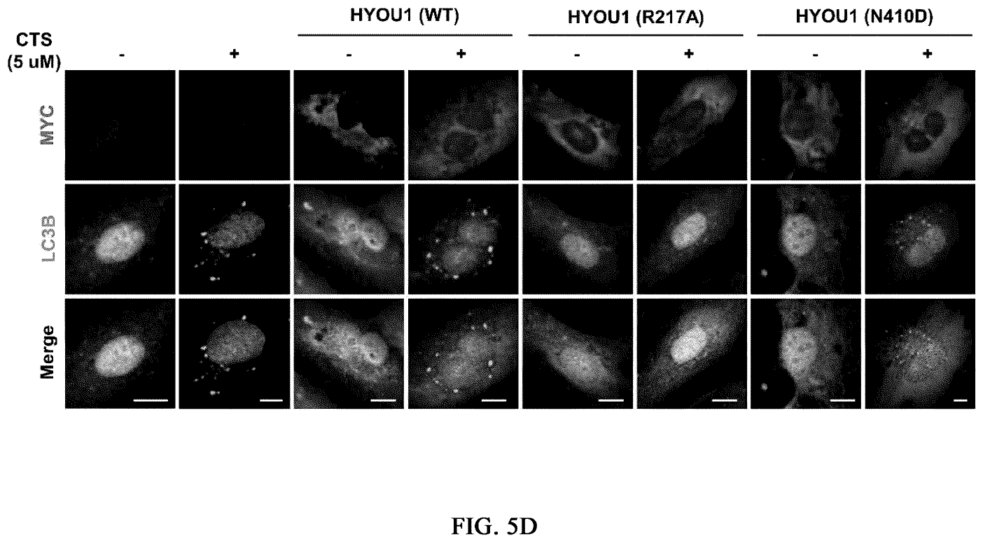

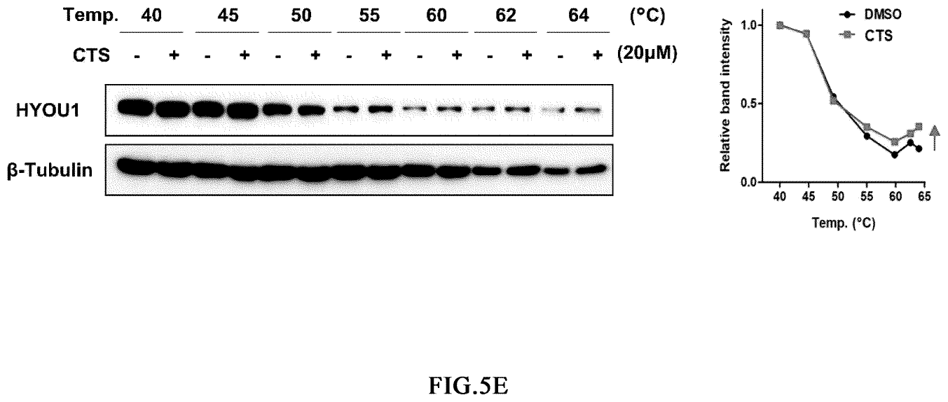

DARTS-western analysis was used to validate HYOU1 as a target protein of CTS in the lysate of HUVECs. Specifically, it was found that the proteolysis of HYOU1 by Pronase in the HUVEC cell lysate was inhibited by CTS (FIG. 5A). In addition, CTS was resistant to degradation by Pronase (protease) in a dose-dependent manner, with an EC₅₀ value calculated to be about 1 μM (FIG. 5B).

In addition, mutant versions of the HYOU1 expression plasmid with key amino acid substitutions, including MYC-HYOU1 (R217A) and MYC-HYOU1 (N410D), were generated to experimentally validate the binding residues for CTS. HEK293 cells were transfected with wild-type MYC-HYOU1, MYC-HYOU1R217A, or MYC-HYOU1N410D vectors for 48 hours, and then DARTS assay was performed using a membrane protein pool. As a result, in the cells transfected with the mutant types of HYOU1, the treatment with CTS did not inhibit the proteolysis of HYOU1 (FIG. 5C). This result indicates that ARG217 and ASN410 are indeed essential for the binding of CTS to HYOU1.

It was also investigated whether the mutant HYOU1-expressing cells had any effect on autophagic activity by not binding to CTS. As a result, a decreased intensity of autophagosomes was observed in the proteins expressing the HYOU1 mutants (FIG. 5D), indicating that ARG217 and ASN410 are hotspots for CTS binding in HYOU1. Furthermore, the reduction in autophagosome intensity suggests that the binding of CTS to HYOU1 is important in promoting autophagy.

As another validation method to determine the targets of small molecules, cellular thermal shift assay (CETSA) was used to observe the pattern of resistance to heat. Specifically, HEK293 cells were treated with DMSO (control group) or CTS and then heated. Soluble proteins were collected after cell lysis, and western blot was performed. Then, the effect of CTS on HYOU1 was determined by CETSA assay. The result showed that CTS inhibited the thermal degradation of HYOU1 when the HEK293 cells were heated (FIG. 5E). This suggests that CTS was directly bound to HYOU1.

Taken together, HYOU1 was identified as a novel target of CTS by DARTS-LC-MS/MS, and it was validated by in silico and various in vitro assays.

### CTS Induces Autophagy to Remove Lipids.

The above experiments confirm that CTS targets HYOU1 to reduce contact between the ER and mitochondria.

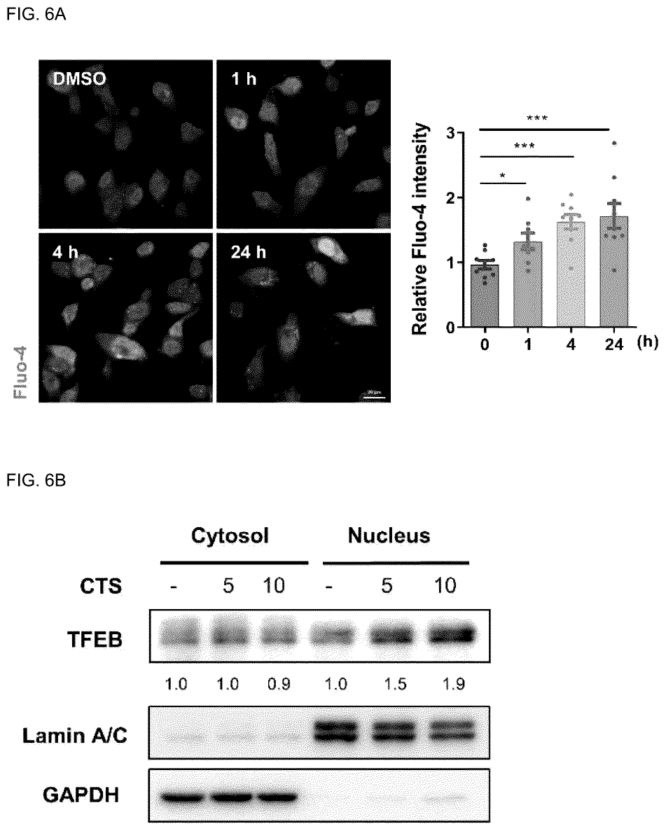

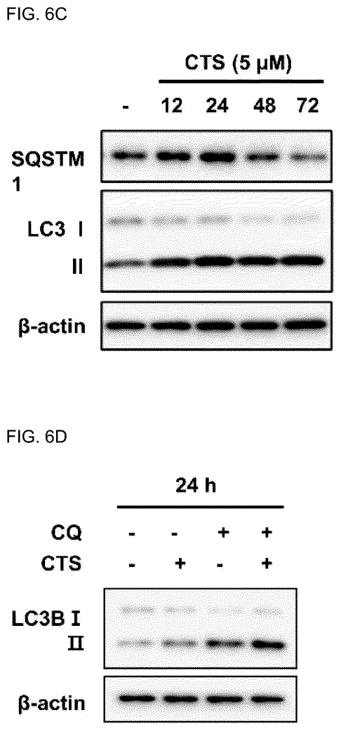

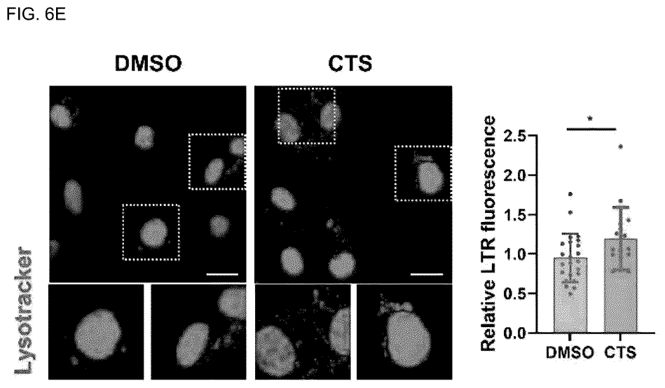

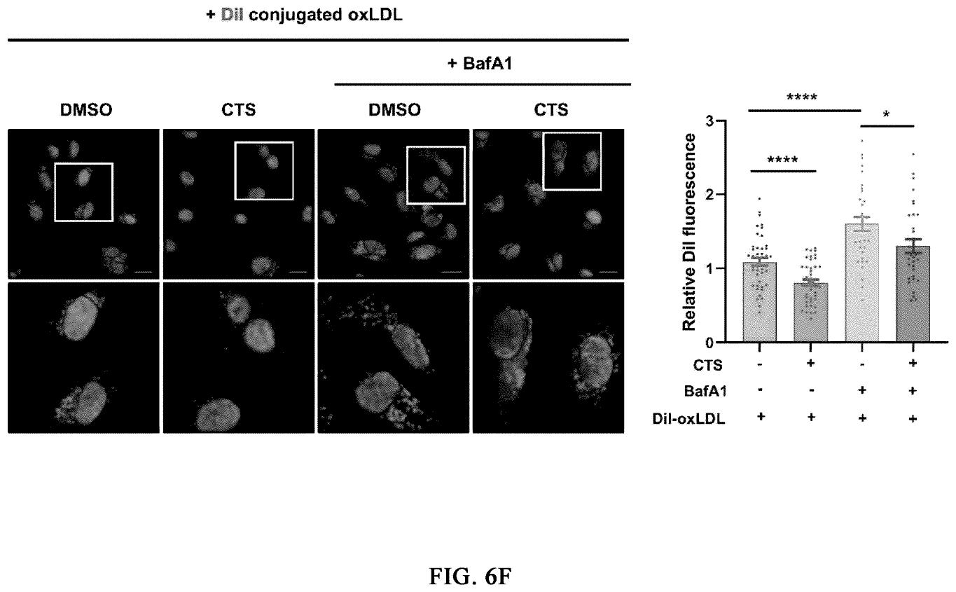

In addition, as a result of labeling the cytosolic calcium of CTS-treated HUVECs with Fluo-4 and observing the fluorescence intensity, it was confirmed that the CTS treatment increased cytosolic calcium (FIG. 6A). This means that more calcium released from the ER is likely to remain in the cytoplasm as the distance between the two cell organelles increases. It was investigated whether calcium release from lysosomes contributed to the increase in cytosolic calcium. As a result of treating cells transfected with GCaMP3-TRPML1, which is a lysosome-targeted genetically encoded Ca²⁺ marker, with CTS, no significant change was observed as compared to DMSO.

The nuclear translocation of TFEB, which is a key transcription factor in lysosomal biosynthesis and autophagy, is regulated by cytosolic calcium. TFEB increases the transcription of various lysosomal genes and promotes autophagy. When HUVECs were treated with CTS and fractionated into the cytoplasm and nucleus, the expression level of TFEB in the nucleus was increased in the CTS-treated cells (FIG. 6B). Furthermore, the change in the CTS-induced intracellular localization of TFEB was investigated by immunofluorescence staining. The result confirmed that the CTS treatment enhanced autophagy by activating the Ca²⁺-TFEB axis. This suggests that the CTS-induced autophagic activity is mediated via TFEB.

Furthermore, after treating HUVECs with CTS, the expression level of LC3-II and SQSTM1 was determined 24, 48 and 72 hours later (FIG. 6C). The result showed that SQSTM1 was decreased and LC3-II was increased by the CTS treatment.

Furthermore, after treating HUVECs with the lysosomal inhibitor chloroquine (CQ) together with CTS, the expression level of LC3-II was investigated (FIG. 6D). The result showed that the cells treated with both CQ and CTS increased the expression level of LC3-II more than in the cells treated with CQ alone. This means that CTS induces autophagic flux.

Furthermore, as a result of staining CTS-treated HUVECs with LysoTracker Red and measuring fluorescence intensity, the increase in LysoTracker Red fluorescence, which is used as an indicator of lysosomal activity, was observed in the CTS-treated HUVECs (FIG. 6E).

Furthermore, as a result of treating HUVECs with Dil-conjugated oxLDL to visualize and quantify intracellular oxLDL level, it was found that the CTS treatment significantly reduced the oxLDL level as compared to the control group (FIG. 6F). Furthermore, as a result of treating HUVECs stimulated with Dil-oxLDL with CTS and BafA1, which is an autophagy flux inhibitor, the ability of CTS to reduce lipid level was attenuated. This suggests that CTS exerts its lipid-lowering effect by promoting autophagy-mediated degradation of excess intracellular lipids.

These results suggest that CTS induces autophagy to degrade excess intracellular lipids, which may be useful in the treatment of diseases characterized by lipid accumulation, such as atherosclerosis.

### The Role of HYOU1 in Autophagy as a Target of CTS

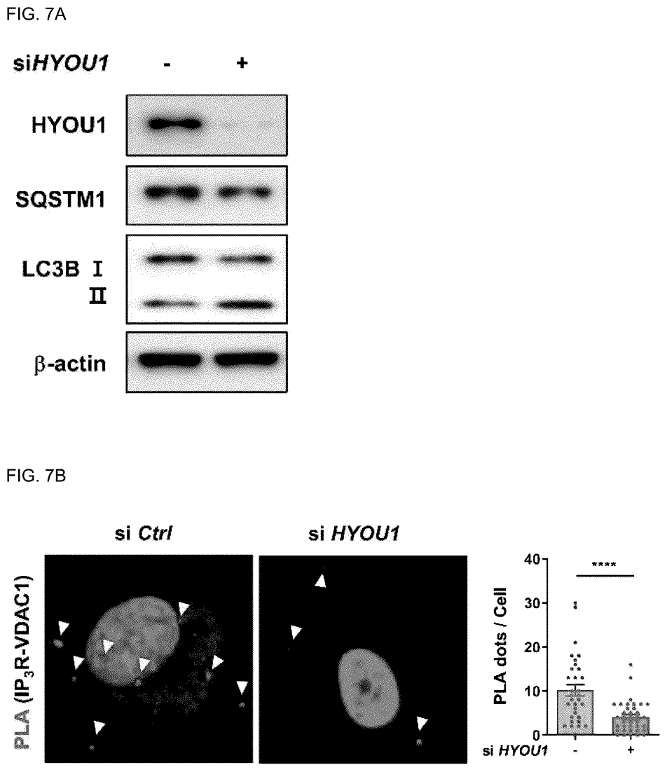

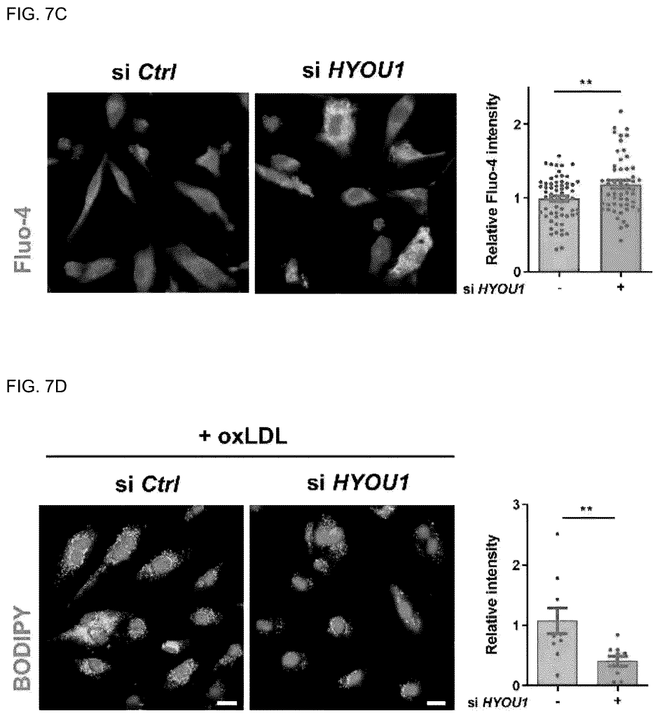

It was investigated whether knockdown of HYOU1 using siRNA would result in CTS-like activity. Specifically, after transfecting HUVECs with si-HYOU1 (20 nM), the expression level of HYOU1, SQSTM1 and LC3 was determined. The result showed that the knockdown of HYOU1 decreased the expression level of SQSTM1 and increased the expression level of LC3-II (FIG. 7A). This indicates autophagic activity.

Furthermore, as a result of performing PLA assay to determine the effect of HYOU1 knockdown on the interaction between ER (IP3R) and mitochondria (VDAC1), it was found that ER-mitochondrial contacts were reduced in the HYOU1-deficient cells (FIG. 7B). This means that the MAM is engaged in autophagic activity.

Furthermore, cytosolic calcium increased (FIG. 7C) and lipid accumulation decreased (FIG. 7D) after the HYOU1 knockdown, confirming that autophagy is promoted by HYOU1 silencing.

This result indicates that HYOU1 is not only a target of CTS, but also plays an important role in linking calcium signaling, autophagy, and lipid metabolism.

### CTS Ameliorates Pulmonary Arterial Hypertension Through Induction of Autophagy in TGF-Treated Pulmonary Artery Cells.

Pulmonary arterial hypertension (PAH) is characterized by increased pressure in the pulmonary arteries, heart failure, etc. Vasoconstriction is caused by the contraction of smooth muscle cells (SMCs), and TGF-β abnormally regulates smooth muscle cells in PAH.

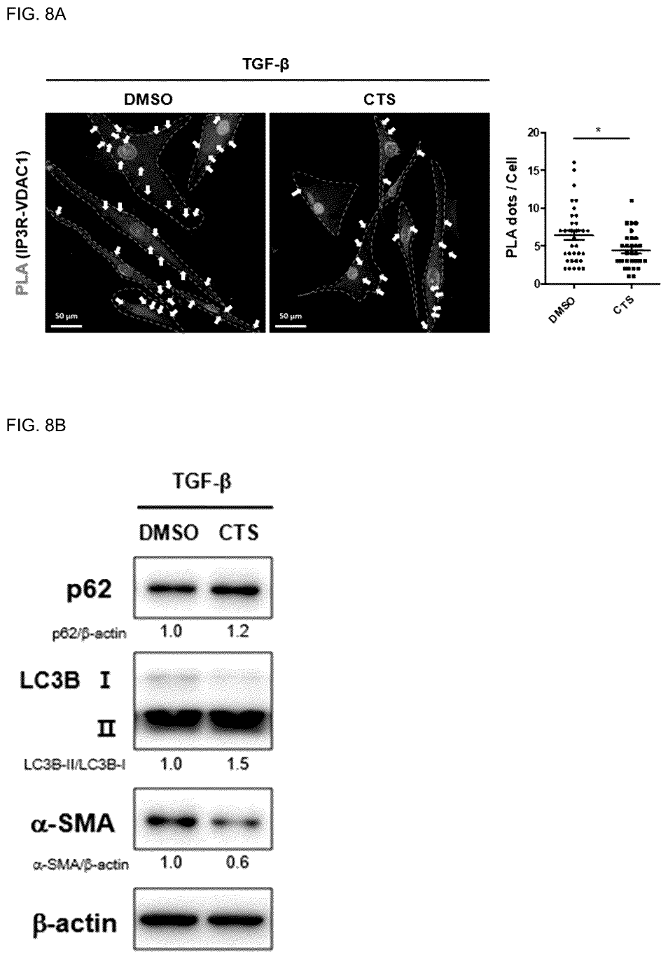

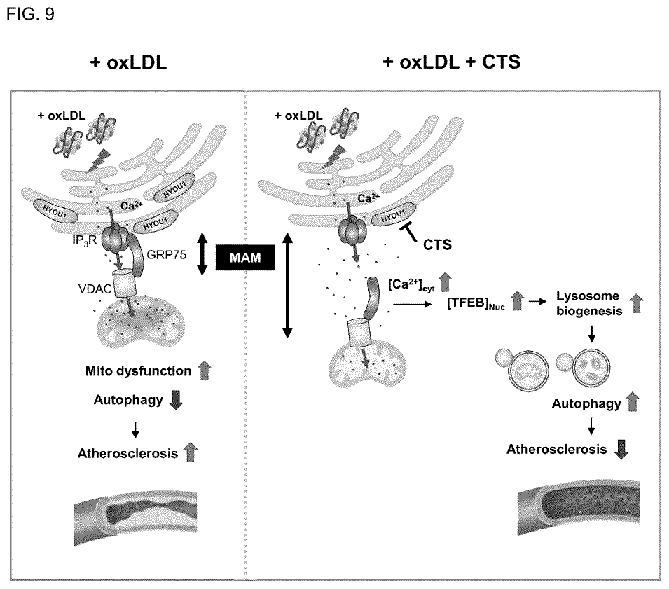

As a result of treating a pulmonary arterial hypertension (PAH) cell model, in which pulmonary artery smooth muscle cells (PASMCs) were treated with TGF-β to induce cell contraction, with CTS (5 M) and performing PLA (IP3R-VDAC1) assay, it was confirmed that the ER-mitochondrial interaction was decreased (FIG. 8A).

In addition, as a result of treating the pulmonary arterial hypertension (PAH) cell model with CTS and investigating the expression level of autophagy markers (p62 and LC3B-II) and a smooth muscle cell proliferation-related marker (α-SMA), it was found that the expression level of the autophagy markers was increased and the expression level of the smooth muscle cell proliferation-related marker was decreased.

This result indicates that the CTS of the present disclosure is effective in alleviating pulmonary arterial hypertension diseases.

### CONCLUSION

The CTS of the present disclosure is a small molecule capable of modulating HYOU1 and has therapeutic efficacy against atherosclerosis and pulmonary arterial hypertension.

Specifically, the CTS of the present disclosure regulates MAM. More specifically, CTS binds to HYOU1, which is an endoplasmic reticulum protein, and disrupts the contact and interaction between the endoplasmic reticulum and mitochondria, which are cell organelles. As a result, calcium released from the endoplasmic reticulum does not enter the mitochondria but remains in the cytoplasm. This leads to an increase in cytosolic calcium levels and activates the TFEB pathway to induce autophagy, which in turn leads to anti-atherosclerotic and/or antihypertensive activity.

While the foregoing has described certain aspects of the present disclosure in detail, it will be apparent to one of ordinary skill in the art that these specific descriptions are merely specific exemplary embodiments and are not intended to limit the scope of the present disclosure. Accordingly, it is to be understood that the substantial scope of the present disclosure is defined by the appended claims and their equivalents.

What is claimed is:

1. A method for treating a disease related to an autophagy disorder, the method comprising administering a HYOU1 (hypoxia-upregulated protein 1) inhibitor to an individual in need thereof or inhibiting expression of a HYOU1 (hypoxia-upregulated protein 1) gene in the individual.

2. The method according to claim 1, wherein the HYOU1 (hypoxia-upregulated protein 1) is a chaperone protein in the endoplasmic reticulum contained in a cell of the individual.

3. The method according to claim 1, wherein the method comprises inhibiting the HYOU1 (hypoxia-upregulated protein 1) protein or inhibiting the expression of the HYOU1 (hypoxia-upregulated protein 1) gene to induce autophagy through one or more of the followings:
1) inhibition of calcium transfer from the endoplasmic reticulum to mitochondria;
2) increase of lysosomal activity;
3) increase of autophagic activity; and
4) increase of distance between the endoplasmic reticulum and mitochondria.

4. The method according to claim 1, wherein the HYOU1 (hypoxia-upregulated protein 1) inhibitor inhibits the interaction of the endoplasmic reticulum and mitochondria in a cell.

5. The method according to claim 4, wherein the HYOU1 (hypoxia-upregulated protein 1) inhibitor is a composition comprising cryptotanshinone (CTS) or a salt thereof and a pharmaceutically acceptable carrier.

6. The method according to claim 1, wherein the autophagy disorder-related disease is a disease caused by accumulation of abnormal proteins induced by reduced intracellular autophagy or a degenerative disease.

7. The method according to claim 6, wherein the caused by accumulation of abnormal proteins or the degenerative disease is any one selected from arteriosclerosis, pulmonary hypertension, Alzheimer's disease, Parkinson's disease, type 2 diabetes, amyotrophic lateral sclerosis, dialysis-related amyloidosis, cystic fibrosis, sickle cell anemia, Huntington's disease, Creutzfeldt-Jakob disease, Lewy body dementia, inclusion body myositis, cerebral amyloid angiopathy, traumatic brain injury, frontotemporal dementia, progressive supranuclear palsy, corticobasal degeneration, Pick's disease, and argyrophilic grain disease.

8. The method according to claim 7, wherein the arteriosclerosis is atherosclerosis.

9. The method according to claim 8, wherein The HYOU1 (hypoxia-upregulated protein 1) inhibitor reduces intracellular lipids by inducing autophagy in atherosclerosis.

10. The method according to claim 7, wherein the pulmonary hypertension is pulmonary arterial hypertension.
